SEQUENCE LISTING (39 sequences)

SEQ ID NO: 1 - DNA, 188 bp, Chlamydomonas reinhardtii
  misc_feature (1)..(188): RbcS2 (ribulose-1,5-bisphosphate carboxylase/oxygenase small subunit 2) 3 prime untranslated region
cgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttg

SEQ ID NO: 2 - DNA, 459 bp, Chlamydomonas reinhardtii
  misc_feature (1)..(459): Beta-2-tubulin (TUB2) 3 prime untranslated region
atgccggcacctccatgcgccactgaacgtgtagcgtgactgtggcggccttggcagttttgaccgtgactgaccctggacaaagggtccctgactgaagacaacttgacatgtgattgccatttgacgctttggtgtggaggcggattgtgagatgggaggggggcccattgccttcgtgaccataacgacatcgaatttcatacatgtgaacagttcagcatggacattcatctcgtcggattagctcttgtgtgataggccatagcagctggactgttgtgggctctcgatctgcgtagctactggctgtgattgtgcttcaggcggcaggggcaggtaactgccctgaacgtaaaggtgcagcagcagacagcggatgtgcagaacgaatagcgcagtggataaggttgatgggtggccacacactcgtgcacgtgtaataagatacacgaatcg

SEQ ID NO: 3 - DNA, 37 bp, Artificial Sequence
  consensus intron splice acceptor intronic sequence
cagcatctaaccctgcgtcgcttttttttttttcagg

SEQ ID NO: 4 - DNA, 31 bp, Artificial Sequence
  intron splice acceptor sequence
ctaaccctgcgtcgcttttttttttttcagg

SEQ ID NO: 5 - DNA, 261 bp, Chlamydomonas reinhardtii
  misc_feature (1)..(261): Hsp70A proximal promoter
cgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcaactcgcttaaga

SEQ ID NO: 6 - DNA, 204 bp, Chlamydomonas reinhardtii
  misc_feature (1)..(204): RbcS2 (ribulose-1,5-bisphosphate carboxylase/oxygenase small subunit 2) proximal promoter
gaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa

SEQ ID NO: 7 - DNA, 176 bp, Chlamydomonas reinhardtii
  misc_feature (1)..(176): Beta-2-tubulin (TUB2) proximal promoter
ttcgaccccccgaagccccttcggggctgcatgggcgctccgtgccgctccagggccagcgctgtttaaatagccaggcccccgattgcaaagacattatagcgagctaccaaagccatattcaaacacctagatcactaccacttctacacaggccactcgagcttgtgatcgca

SEQ ID NO: 8 - DNA, 9 bp, Artificial Sequence
  consensus intron splice donor sequence
maggtragt

SEQ ID NO: 9 - DNA, 50 bp, Streptomyces actinophage R4
  misc_feature: R4 attB site in direct orientation
gagttgcccatgaccatgccgaagcagtggtagaagggcaccggcagaca

SEQ ID NO: 10 - DNA, 50 bp, Streptomyces actinophage R4
  misc_feature: attB site in reverse (inverted) orientation
tgtctgccggtgcccttctaccactgcttcggcatggtcatgggcaactc

SEQ ID NO: 11 - DNA, 33 bp, Zygosaccharomyces bisporus
  misc_feature: B3 recombinase recognition target sequence
ggttgcttaagaataagtaattcttaagcaacc

SEQ ID NO: 12 - DNA, 48 bp, Saccharomyces cerevisiae 2 micron plasmid
  misc_feature: FRT WT
gaagttcctattccgaagttcctattctctagaaagtataggaacttc

SEQ ID NO: 13 - DNA, 48 bp, Artificial Sequence
  FRT3 sequence
gaagttcctattccgaagttcctattcttcaaatagtataggaacttc

SEQ ID NO: 14 - DNA, 48 bp, Artificial Sequence
  FRT5 sequence
gaagttcctattccgaagttcctattcttcaaaaggtataggaacttc

SEQ ID NO: 15 - DNA, 50 bp, Streptomyces actinophage TG1
  misc_feature: TG1 recombinase attB sequence
tcgatcagctccgcgggcaagaccttctccttcacggggtggaaggtcgg

SEQ ID NO: 16 - DNA, 49 bp, Streptomyces actinophage TG1
  misc_feature: TG1 recombinase attP sequence
gtccagcccaacagtgttagtctttgctcttacccagttgggcgggata

SEQ ID NO: 17 - DNA, 39 bp, Zygosaccharomyces bailii
  misc_feature: B2 recombinase recognition sequence
gagtttcattaaggaataactaattccctaatgaaactc

SEQ ID NO: 18 - DNA, 35 bp, Zygosaccharomyces fermentati
  misc_feature: SM1 recombinase recognition sequence
gaaatggaaaggaatggttcattcctttccatttc

SEQ ID NO: 19 - DNA, 31 bp, Zygosaccharomyces rouxii
  misc_feature: SR1 recombinase recognition sequence
ttgatgaaagaatacgttattctttcatcaa

SEQ ID NO: 20 - DNA, 35 bp, Kluyveromyces lactis
  misc_feature: KD1 recombinase recognition sequence
catttgtctgataatgaagcattatcagacaaatg

SEQ ID NO: 21 - DNA, 145 bp, Chlamydomonas reinhardtii
  misc_feature: RbcS2 (ribulose-1,5-bisphosphate carboxylase/oxygenase small subunit 2)intron 1
gtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcag

SEQ ID NO: 22 - protein, 7 aa, Artificial Sequence
  2A peptide consensus sequence
DXENPGP

SEQ ID NO: 23 - protein, 18 aa, Thosea asigna
  MISC_FEATURE: Thoseaasigna virus 2A peptide
EGRGSLLTCGDVEENPGP

SEQ ID NO: 24 - protein, 469 aa, Streptomyces actinophage R4
  MISC_FEATURE: (sre)R4 recombinase
MNRGGPTVRADIYVRISLDRTGEELGVERQEESCRELCKSLGMEVGQVWVDNDLSATKKNVVRPDFEAMIASNPQAIVCWHTDRLIRVTRDLERVIDLGVNVHAVMAGHLDLSTPAGRAVARTVTAWATYEGEQKAERQKLANIQNARAGKPYTPGIRPFGYGDDHMTIVTAEADAIRDGAKMILDGWSLSAVARYWEELKLQSPRSMAAGGKGWSLRGVKKVLTSPRYVGRSSYLGEVVGDAQWPPILDPDVYYGVVAILNNPDRFSGGPRTGRTPGTLLAGIALCGECGKTVSGRGYRGVLVYGCKDTHTRTPRSIADGRASSSTLARLMFPDFLPGLLASGQAEDGQSAASKHSEAQTLRERLDGLATAYAEGAISLSQMTAGSEALRKKLEVIEADLVGSAGIPPFDPVAGVAGLISGWPTTPLPTRRAWVDFCLVVTLNTQKGRHASSMTVDDHVTIEWRDVAE

SEQ ID NO: 25 - protein, 567 aa, Zygosaccharomyces bisporus
  MISC_FEATURE: B3 recombinase
MSSYMDLVDDEPATLYHKFVECLKAGENFCGDKLSGIITMAILKAIKALTEVKKTTFNKYKTTIKQGLQYDVGSSTISFVYHLKDCDELSRGLSDAFEPYKFKIKSNKEATSFKTLFRGPSFGSQKNWRKKEVDREVDNLFHSTETDESIFKFILNTLDSIETQTNTDRQKTVLTFILLMTFFNCCRNNDLMNVDPSTFKIVKNKFVGYLLQAEVKQTKTRKSRNIFFFPIRENRFDLFLALHDFFRTCQPTPKSRLSDQVSEQKWQLFRDSMVIDYNRFFRKFPASPIFAIKHGPKSHLGRHLMNSFLHKNELDSWANSLGNWSSSQNQRESGARLGYTHGGRDLPQPLFGFLAGYCVRNEEGHIVGLGLEKDINDLFDGIMDPLNEKEDTEICESYGEWAKIVSKDVLIFLKRYHSKNACRRYQNSTLYARTFLKTESVTLSGSKGSEEPSSPVRIPILSMGKASPSEGRKLRASEHANDDNEIEKIDSDSSQSEEIPIEMSDSEDETTASNISGIYLDMSKANSNVVYSPPSQTGRAAGAGRKRGVGGRRTVESKRRRVLAPIN

SEQ ID NO: 26 - protein, 423 aa, Saccharomyces cerevisiae 2 micron plasmid
  MISC_FEATURE: Flp recombinase
MSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 27 - protein, 594 aa, bacteriophage phiBT1
  MISC_FEATURE: phiBT1 recombinase
MSPFIAPDVPEHLLDTVRVFLYARQSKGRSDGSDVSTEAQLAAGRALVASRNAQGGARWVVAGEFVDVGRSGWDPNVTRADFERMMGEVRAGEGDVVVVNELSRLTRKGAHDALEIDNELKKHGVRFMSVLEPFLDTSTPIGVAIFALIAALAKQDSDLKAERLKGAKDEIAALGGVHSSSAPFGMRAVRKKVDNLVISVLEPDEDNPDHVELVERMAKMSFEGVSDNAIATTFEKEKIPSPGMAERRATEKRLASVKARRLNGAEKPIMWRAQTVRWILNHPAIGGFAFERVKHGKAHINVIRRDPGGKPLTPHTGILSGSKWLELQEKRSGKNLSDRKPGAEVEPTLLSGWRFLGCRICGGSMGQSQGGRKRNGDLAEGNYMCANPKGHGGLSVKRSELDEFVASKVWARLRTADMEDEHDQAWIAAAAERFALQHDLAGVADERREQQAHLDNVRRSIKDLQADRKPGLYVGREELETWRSTVLQYRSYEAECTTRLAELDEKMNGSTRVPSEWFSGEDPTAEGGIWASWDVYERREFLSFFLDSVMVDRGRHPETKKYIPLKDRVTLKWAELLKEEDEASEATERELAAL

SEQ ID NO: 28 - protein, 619 aa, Streptomyces actinophage TG1
  MISC_FEATURE: TG1 recombinase
MVILAGGYDRQSAERENSSTASPATQRAANRGKAEALAKEYARDGVEVKWLGHFSEAPGTSAFTGVDRPEFNRILDMCRNREMNMIIVHYISRLSREEPLDIIPVVTELLRLGVTIVSVNEGTFRPGEMMDLIHLIMRLQASHDESKNKSVAVSNAKELAKRLGGHTGSTPYGFDTVEEMVPNPEDGGKLVAIRRLVPSAHTWEGAHGSEGAVIRWAWQEIKTHRDTPFKGGGAGSFHPGSLNGLCERLYRDKVPTRGTLVGKKRAGSDWDPGVLKRVLSDPRIAGYQADIAYKVRADGSRGGFSHYKIRRDPVTMEPLTLPGFEPYIPPAEWWELQEWLQGRGRGKGQYRGQSLLSAMDVLYCYGSGQLDPETGYSNGSTMAGNVREGDQAHKSSYACKCPRRVHDGSSCSITMHNLDPYIVGAIFARITAFDPADPDDLEGDTAALMYEAARRWGATHERPELKGQRSELMAQRADAVKALEELYEDKRNGGYRSAMGRRAFLEEEAALTLRMEGAEERLRQLDAADSPVLPIGEWLGDRGSDPTGPGSWWALAPLEDRRAFVRLFVDRIEVIKLPKGVQRPGRVPPIADRVRIHWAKPKVEEETEPETLNGFTAAA

SEQ ID NO: 29 - protein, 515 aa, Zygosaccharomyces bailii
  MISC_FEATURE: B2 recombinase
MSEFSELVRILPLDQVAEIKRILSRGDPIPLQRLASLLTMVILTVNMSKKRKSSPIKLSTFTKYRRNVAKSLYYDMSSKTVFFEYHLKNTQDLQEGLEQAIAPYNFVVKVHKKPIDWQKQLSSVHERKAGHRSILSNNVGAEISKLAETKDSTWSFIERTMDLIEARTRQPTTRVAYRFLLQLTFMNCCRANDLKNADPSTFQIIADPHLGRILRAFVPETKTSIERFIYFFPCKGRCDPLLALDSYLLWVGPVPKTQTTDEETQYDYQLLQDTLLISYDRFIAKESKENIFKIPNGPKAHLGRHLMASYLGNNSLKSEATLYGNWSVERQEGVSKMADSRYMHTVKKSPPSYLFAFLSGYYKKSNQGEYVLAETLYNPLDYDKTLPITTNEKLICRRYGKNAKVIPKDALLYLYTYAQQKRKQLADPNEQNRLFSSESPAHPFLTPQSTGSSTPLTWTAPKTLSTGLMTPGEEGSHGFPPEVEEQDDGTLPMSCAQESGMDRHPAACASARINV

SEQ ID NO: 30 - protein, 372 aa, Zygosaccharomyces fermentati
  MISC_FEATURE: SM1 recombinase
MATFSKLSERKRSTFIKYSREIRQSVQYDREAQIVKFNYHLKRPHELKDVLDKTFAPIVFEVSSTKKVESMVELAAKMDKVEGKGGHNAVAEEITKIVRADDIWTLLSGVEVTIQKRAFKRSLRAELKYVLITSFFNCSRHSDLKNADPTKFELVKNRYLNRVLRVLVCETKTRKPRYIYFFPVNKKTDPLIALHDLFSEAEPVPKSRASHQKTDQEWQMLRDSLLTNYDRFIATHAKQAVFGIKHGPKSHLGRHLMSSYLSHTNHGQWVSPFGNWSAGKDTVESNVARAKYVHIQADIPDELFAFLSQYYIQTPSGDFELIDSSEQPTTFINNLSTQEDISKSYGTWTQVVGQDVLEYVHSYAMGKLGIRK

SEQ ID NO: 31 - protein, 490 aa, Zygosaccharomyces rouxii
  MISC_FEATURE: R/RS recombinase
MQLTKDTEISTINRQMSDFSELSQILPLHQISKIKDILENENPLPKEKLASHLTMIILMANLASQKRKDVPVKRSTFLKYQRSISKTLQYDSSTKTVSFEYHLKDPSKLIKGLEDVVSPYRFVVGVHEKPDDVMSHLSAVHMRKEAGRKRDLGNKINDEITKIAETQETIWGFVGKTMDLIEARTTRPTTKAAYNLLLQATFMNCCRADDLKNTDIKTFEVIPDKHLGRMLRAFVPETKTGTRFVYFFPCKGRCDPLLALDSYLQWTDPIPKTRTTDEDARYDYQLLRNSLLGSYDGFISKQSDESIFKIPNGPKAHLGRHVTASYLSNNEMDKEATLYGNWSAAREEGVSRVAKARYMHTIEKSPPSYLFAFLSGFYNITAERACELVDPNSNPCEQDKNIPMISDIETLMARYGKNAEIIPMDVLVFLSSYARFKNNEGKEYKLQARSSRGVPDFPDNGRTALYNALTAAHVKRRKISIVVGRSIDTS

SEQ ID NO: 32 - protein, 447 aa, Kluyveromyces lactis
  MISC_FEATURE: KD1 recombinase
MSTFAEAAHLTPHQCANEINEILESDTFNINAKEIRNKLASLFSILTMQSLSIRREMKINTYRSYKSAIGKSLSFDKDDKIIKFTVRLRKTESLQKDIESALPSYKVVVSPFKNQEVSLFDRYEETHKYDASMVGLQFTNILSKEKDIWKIVSRIACFFDQSCVTTTKRAEYRLLLLGAVGNCCRYSDLKNLDPRTFEIYNNSFLGPIVRATVTETKSRTERYVNFYPVNGDCDLLISLYDYLRVCSPIEKTVSSNRPTNQTHQFLPESLARTFSRFLTQHVDEPVFKIWNGPKSHFGRHLMATFLSRSEKGKYVSSLGNWAGDREIQSAVARSHYSHGSVTVDDRVFAFISGFYKEAPLGSEIYVLKDPSNKPLSREELLEEEGNSLGSPPLSPPSSPRLVAQSFSAHPSLQLFEQWHGIISDEVLQFIAEYRRKHELRSQRTVVA

SEQ ID NO: 33 - DNA, 60 bp, Artificial Sequence
  oligo-dT adaptor primer
gccctaggcgagaacgagatctagctctagaattcggacgttttttttttttttttttvn

SEQ ID NO: 34 - DNA, 51 bp, Artificial Sequence
  “SacII′ anchor primer
gagatctagctctagaattcggacgnnnnnnnnnnnnnnnnnnnnnngcgg

SEQ ID NO: 35 - DNA, 51 bp, Artificial Sequence
  EagI prime anchor primer
gagatctagctctagaattcggacgnnnnnnnnnnnnnnnnnnnnnngccg

SEQ ID NO: 36 - DNA, 51 bp, Artificial Sequence
  SmaI/XmaI prime anchor primer
gagatctagctctagaattcggacgnnnnnnnnnnnnnnnnnnnnnncggg

SEQ ID NO: 37 - protein, 150 aa, Scheffersomyces stipites
  MISC_FEATURE: cytosine deaminase (codA)
MPFNDKKGMQIALEEAKKGYEEGGVPIGGALISEDGTVLGRGHNMRFQKDSAILHGEMSVLENAGRLKGSVYKNCTMYTTLSPCHMCSGACLMYGIKRVVLGENVNFVGAEALLRSEGVEVVNLNDPECKALMKKFIDERPEDWFEDIGE

SEQ ID NO: 38 - protein, 153 aa, Torulaspora delbrueckii
  MISC_FEATURE: cytosine deaminase (codA)
MSNQWDKIGMDVAYEEALKGFAQGGVPIGGCLINNKDGTILGRGHNMRFQKGSATLHGEISTLENCGRLPGKVYKDTTLYTTLSPCDMCTGAIIMYGIPRCVIGENVNFKSPGEQYLQSRGHEVVVVDDERCKAIMKKLIDERPQDWFEDIGE

SEQ ID NO: 39 - DNA, 330 bp, Phaeodactylum tricornutum
  misc_feature: fcpA terminator
cagaagcgtgctatcgaactcaaccagggacgtgcggcacaaatgggcatccttgctctcatggtgcacgaacagttgggagtctctatccttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgag

(19) United States
(12) Patent Application Publication
(10) Pub. No.: US 2015/0344895 A1
(43) Pub. Date: Dec. 3, 2015

(54) METHODS OF MODIFYING ALGAL CELL GENOMES

(71) Applicant: Spicer Consulting Ltd, Bedfordshire (GB)

(72) Inventor: Andrew Spicer, Bedfordshire (GB)

(73) Assignee: Spicer Consulting Ltd, Bedfordshire (GB)

(21) Appl. No.: 14/421,884

(22) PCT Filed: Aug. 16, 2013
(86) PCT No.: PCT/EP2013/002480

§ 371 Date: Feb. 16, 2015

(30) Foreign Application Priority Data
Aug. 16, 2012 (GB) 1214645.2

(51) Int. Cl. C12N 15/82; C12Q 1/04

(52) U.S. Cl. CPC C12N 15/82; C12Q 1/04

(57) ABSTRACT

A method of introducing a target gene sequence into a primed algal cell, comprising a step of providing a primed algal cell comprising providing an algal cell; introducing into the algal cell an integration cassette comprising at least two site-specific recombination sites and at least one selectable marker gene wherein the at least two site-specific recombination sites are positioned to flank the at least one selectable marker gene; and selecting cells which have incorporated the integration cassette by cultivating the cells in a selective media and selecting growing cells, wherein an ability of the cells to be cultured on the selective media is dependent on a presence of the at least one selectable marker in a genome of the algal cell. The site-specific recombination sites may be compatible. The method includes a further step of effecting targeted site-specific recombinase mediated deletion of the target cassette can be carried out. The site-specific recombination sites maybe heterospecific to permit introduction of target gene(s). The method further comprises a step of providing a target cassette comprising at least one target gene sequence flanked by a type I site-specific recombination site and a type II site-specific recombination site, these sites being capable of recombining with those in the primed algal cell. Moreover, the method includes a further step of effecting targeted site-specific recombinase mediated insertion of the target cassette into the algal genome by effecting recombination between corresponding type I and type II site-specific recombination sites flanking the target gene sequence and located in the algal genome, such that the target gene sequence is introduced into the algal genome replacing the at least one selectable marker. The invention also concerns integration cassettes for use in the method above, as well as an algal cell for use with and modified algal cell produced by the above method.

### TECHNICAL FIELD

The present invention relates to methods of modifying cell genomes, for example to methods of modifying algal cell genomes, for example in association with transgenic algal technology.

### BACKGROUND

Modification of algal genomes, and in particular microalgal genomes, has, to date, proven to be challenging for several reasons. First, transformation efficiencies tend to be low for many strains of algae, making it difficult to reproducibly insert new genes into a microalgal genome. Second, when genes are able to be integrated into an algal genome, non-homologous integration is markedly favoured over homologous integration into nuclear DNA; in other words, the location of an integrated gene within a genome cannot be predicted or directed. Difficulties in generating specifically engineered algal cells are further compounded by a limited availability of genomic sequence data for many more relevant algal, microalgal and macroalgal strains. In addition, transgene expression in algae is often reduced or silenced within a comparatively short period of time, making exploitation of the integrated genetic characteristics difficult to sustain.

The lack of genomic sequence data and the rapid reduction or silencing of transgene expression represent a substantial barrier to the application of algal, microalgal and macroalgal metabolic engineering because established promoters and regulatory elements are not presently available. Whereas heterologous sequences from other species can be used such use often leads to rapid transgene silencing. For this reason, a standardised approach for providing a controlled modification of algal, microalgal and macroalgal genomes, and in particular for providing an approach to predictable and directed insertion of genes into an algal, microalgal or macroalgal genome, is an attractive proposition.

A standardised approach for removing selectable marker genes that have been inserted into the algal genome would also be extremely useful. This is a particularly important consideration for any genetically modified algal strain that might be utilised in large-scale production, especially in an outdoor environment, as there are only a limited number of selectable marker genes that are permitted to remain within genetically modified crop-plants that are grown in outdoor environments; it is thought that a similar level of regulation will apply to genetically modified algae, and in particular microalgae. A standardised approach for removing randomly inserted heterologous transgenes would also be extremely useful.

In view of the challenges described above, contemporary transgenic algal technology is presently limited to the design of expression vectors containing one of a small group of characterised algal promoters and one of a limited set of suitable selectable marker genes, with successful transformation determined by subsequent selection for the presence of the selectable marker gene. A gene of interest, when included within the expression vector, can be presumed to have been incorporated into the algal genome with the selectable marker gene. Using this technique, the transgene DNA of the expression vector is incorporated at random into the algal genome, with its site of insertion being largely uncontrolled. This makes it extremely difficult to investigate the relative impact of the insertion of multiple different transgenes because the chances of obtaining two strains with identical positions of insertion is extremely unlikely, and each strain will, therefore, have inserted the transgene DNA encoded by the expression vector at a different location. Different locations of integration can be determined by standard techniques, representing a large burden of experimentation. The differing locations will influence numerous factors including the level of expression and the copy number.

One potential alternative is to employ a process of homologous recombination to insert defined transgenes at specific sites. However, the utility of this method is limited because the efficiency of this process is extremely low for those algae that have been reported to date, particularly for microalgae. Since a first paper describing this methodology was published by Sodeinde and Kindle in 1993 (Sodeinde, O. A. and Kindle, K. L. (1993) Homologous recombination in the nuclear genome of Chlamydomonas reinhardtii. Proc Natl Acad Sci USA. 90(19):9199-9203), only six subsequent publications have described the use of the process of homologous recombination in microalgae (Gumpel N J, Rochaix J D, Purton S. (1994) Studies on homologous recombination in the green alga Chlamydomonas reinhardtii. Curr. Genet. 26(5-6):438-442; Nelson J A, Lefebvre P A. (1995) Targeted disruption of the NIT8 gene in Chlamydomonas reinhardtii. Mol. Cell. Biol. 15(10):5762-5769; Dawson H N, Burlingame R, Cannons A C. (1997) Stable Transformation of Chlorella: Rescue of nitrate reductase-deficient mutants with the nitrate reductase gene. Curr Microbiol. 35(6):356-362; Zorin B, Hegemann P, Sizova I. (2005) Nuclear-gene targeting by using single-stranded DNA avoids illegitimate DNA integration in Chlamydomonas reinhardtii. Eukaryot. Cell. 4(7):1264-1272; Zorin B, Lu Y, Sizova I, Hegemann P (2009) Nuclear gene targeting in Chlamydomonas as exemplified by disruption of the PHOT gene. Gene. 432(1-2):91-96; Kilian O, Benemann C S E, Niyogi K K, Vick B. (2011) High-efficiency homologous recombination in the oil-producing alga Nannochloropsis sp. Proc Natl Acad Sci USA. 108(52):21265-21269), attesting to the current difficulties with this technology. Indeed, all of the reports to date except one (Zorin B, Lu Y, Sizova I, Hegemann P (2009) Nuclear gene targeting in Chlamydomonas as exemplified by disruption of the PHOT gene. Gene. 432(1-2):91-96) where homologous recombination has successfully been used in microalgae have targeted genes encoding products responsible for a selectable phenotype (e.g. auxotrophy for nitrogen or arginine) which can be used, in combination with a conventional selectable marker gene (e.g. antibiotic resistance), to select for successful integration of the transgene.

It is clear that new methods of genetically manipulating algae, microalgae and macroalgae are required. Such methods would be useful for the insertion and removal of target genes preferably in a predictable and directed manner and would be generally applicable across algal, and particularly microalgal strains.

### SUMMARY

The present invention seeks to provide an improved method of genetically manipulating algae, and in particular microalgae.

The improved method is provided as defined in appended claim 1; the improved method utilizing a gene trap optionally combined with a gene replacement process.

The improved method is capable of overcoming many of the deficiencies associated with currently known available methods.

### Gene Trap:

The present invention utilizes a gene trap priming method. Moreover, the gene trap method described herein incorporates site-specific recombination sites into an algal cell genome, enabling an initially trapped genomic site to act as a target for a directed integration of subsequent target nucleic acids into the algal cell genome. This method can be used to effect gene replacement using a target cassette encoding a target gene or genes, or gene stacking of multiple target genes into a trapped site to build up a tandem gene array. The method can also be applied to remove a selectable marker gene which has previously been introduced into an algal genome.

In one aspect, the invention relates to a method of modifying an algal cell genome comprising:
a) incorporating an integration cassette comprising two site-specific recombination sites and a selectable marker gene, wherein the two site-specific recombination sites are positioned to flank the selectable marker gene, into the algal cell genome; and
b) selecting cells which have incorporated the integration cassette by cultivating the cells in a selective media and selecting growing cells, wherein an ability of the cells to be cultured on the selective media is dependent on a presence of the at least one selectable marker in a genome of the algal cell.

Herein the term “flanked” denotes the positioning of the selectable marker gene between the two site-specific recombination sites. This term is not limited to direct flanking, and does not preclude the presence of additional sequences between each site-specific recombination site and the selectable marker gene. The term ‘selectable marker’ refers to one, two or more selectable markers.

In some embodiments, the selected cells may have incorporated the integration cassette within an actively expressed gene. Throughout, the term gene refers to coding and non coding sequences that contribute to the expression and production of a polypeptide and can include introns, exons, promoter sequences and untranslated regions.

### Promoter Trap:

A promoter trap is beneficially employed when implementing the present invention. One specific form of a gene trap described herein is the promoter trap. Here, an integration vector is incorporated into an algal genome such that an endogenous algal promoter becomes actively coupled to an incorporated integration cassette, and drives expression of a selectable marker gene, and subsequent expression of any inserted target genes. In an embodiment, the integration cassette harnesses the function of the endogenous promoter and transcription start site, enabling expression of the selectable marker gene, and subsequent expression of any inserted target genes. Preferably, the integration cassette is incorporated into the algal cell genome such that the promoter of an actively expressed endogenous gene is harnessed.

Herein, throughout the description of embodiments of the disclosure, the term “promoter” includes the promoter itself, and any associated regulatory elements such as the transcription start site.

In this embodiment, the integration cassette further comprises a 3′ untranslated region (3′ UTR) sequence positioned such that one of the site-specific recombination sites is flanked by the 3′ UTR sequence and the selectable marker gene. The arrangement of this construct is depicted in FIG. 1B. The provision of a 3′ UTR from the integration cassette means that there is a need only to harness an endogenous promoter from the algal genome in order for the selectable marker gene, and any one or more subsequently inserted target gene(s), to be expressed.

Herein, throughout the description of embodiments specification, the term “3′ UTR” encompasses the sequence positioned 3′ to an expressed gene and which, preferably, includes a functional polyadenylation signal.

The 3′ UTR may be from an algal gene. In one embodiment, the 3′ UTR may be from a Chlamydomonas reinhardtii gene, such as RbcS2 (SEQ ID NO:1) or beta-tubulin (SEQ ID NO:2).

Within this embodiment, the integration cassette also comprises an intron splice acceptor sequence positioned such that one of the site-specific recombination sites is flanked by the intron splice acceptor sequence and the selectable marker gene. This arrangement is depicted in FIG. 1B. The intron splice acceptor sequence may be a consensus intron splice acceptor sequence or an endogenous intron splice acceptor sequence from any algal or microalgal species. The consensus intron splice acceptor sequence preferably has the sequence (SEQ ID NO:3). In a preferred embodiment, the consensus intron splice acceptor sequence may have the sequence (SEQ ID NO:4).

### PolyA Trap:

A polyA trap constitutes another specific embodiment of the present invention. Here, an integration vector is incorporated into an algal genome such that an endogenous 3′ UTR becomes actively coupled to an incorporated integration cassette, and facilitates expression of a selectable marker gene, and any one or more subsequently inserted target genes. The integration cassette may be incorporated into the algal cell genome such that the 3′ UTR of an actively expressed endogenous gene is harnessed.

In this embodiment, the integration cassette further comprises an algal promoter sequence positioned such that one of the site-specific recombination sites is flanked by the algal promoter sequence and the selectable marker gene. This arrangement is depicted in FIG. 1A.

The algal promoter may be a promoter from any species of algae or microalgae. Particularly preferred promoters are those from Chlamydomonas reinhardtii, Chlorella species including Chlorella vulgaris, Dunaliella salina and Haematococcus pluvialis.

The algal promoter may optionally be a constitutive algal promoter. A constitutive promoter is preferred because it is more likely to allow sustained expression of the selectable marker gene and any one or more target genes subsequently inserted into the algal genome. In one embodiment, the promoter may be selected from a group consisting of the Hsp70A promoter (SEQ ID NO:5), the RbcS2 promoter (SEQ ID NO:6) and the beta-2-tubulin (TUB2) promoter (SEQ ID NO:7). More than one algal promoter (e.g. two, three, four, five or more) may be provided in tandem within the integration cassette. Commonly, two algal promoters are provided in tandem. Preferred tandem combinations of algal promoters are the Chlamydomonas reinhardtii Hsp70A and the RbcS2 promoters, and the Chlamydomonas reinhardtii Hsp70A and the beta-2-tubulin (TUB2) promoters. These pairs of promoters are provided most typically in the orientation where the Hsp70A promoter sequence is positioned immediately upstream (5′) of the RbcS2 or the TUB2 promoter.

Within this embodiment, the integration cassette may also comprise an intron splice donor sequence positioned such that the intron splice donor sequence is flanked by one of the site-specific recombination sites and the selectable marker gene. This arrangement is depicted in FIGS. 1A and 2A. The intron splice donor sequence may be an endogenous or consensus intron splice donor sequence. The consensus intron splice donor sequence preferably has the sequence (SEQ ID NO:8).

Within this embodiment, the integration cassette may also comprise a 5′ untranslated region (5′ UTR) sequence positioned downstream of the promoter.

Combined Promoter and polyA Trap:

In one embodiment, a gene trap is a combined promoter and polyA trap, and utilises an endogenous promoter and an endogenous 3′ UTR from an algal genome to drive expression of a selectable marker gene, and any one or more subsequently inserted target genes. Functional expression of the selectable marker gene, and any one or more subsequently inserted target genes, is therefore dependent upon insertion of the integration cassette into the algal genome such that it acquires the activity of both a promoter as well as a 3′ UTR from the algal genome. Preferably, the integration cassette is incorporated into the algal cell genome such that the promoter and/or 3′ UTR of an actively expressed endogenous gene is harnessed. The promoter and 3′ UTR may be from the same endogenous gene or, less commonly, from mutually different endogenous genes that are positioned in tandem within the algal genome, indicating that a deletion event has occurred at the integration site.

This embodiment allows actively expressed algal genes to be identified, since expression of the selectable marker gene is presumed to be a reflection of the natural expression of the endogenous gene that will have been effectively disrupted.

Within this embodiment, the integration cassette also comprises an intron splice acceptor sequence positioned such that one of the site-specific recombination sites is flanked by the intron splice acceptor sequence and the selectable marker gene. This arrangement is depicted in FIG. 1C. The intron splice acceptor sequence may be a consensus intron splice acceptor sequence or an endogenous intron splice acceptor sequence from any algal or microalgal species. The consensus intron splice acceptor sequence preferably has the sequence (SEQ ID NO:3). In a preferred embodiment, the consensus intron splice acceptor sequence may have the sequence (SEQ ID NO:4).

Within this embodiment, the integration cassette may also comprise an intron splice donor sequence positioned such that the intron splice donor sequence is flanked by one of the site-specific recombination sites and the selectable marker gene. This arrangement is depicted in FIG. 1C. The intron splice donor sequence may be an endogenous or consensus intron splice donor sequence. The consensus intron splice donor sequence preferably has the sequence (SEQ ID NO:8).

### Components of the Integration Cassette:

physical components of the integration cassette will be described in more detail below.

### Selectable Maker Genes:

The selectable marker gene is any gene, the expression of which can be detected as an indication that the integration cassette has been inserted into the algal genome. In some embodiments, expression of the selectable marker gene may indicate that insertion is within an actively expressed algal gene.

The selectable marker gene is preferably a positive selectable marker gene. A positive selectable marker gene is a gene which, upon expression in a cell, imparts a measurable phenotypic property to the cell. Herein, the positive selectable marker gene may be a gene which confers resistance to antibiotic or herbicide. The positive selectable marker gene may confer, for example, resistance to an antibiotic selected from the group consisting of hygromycin B (such as the hph gene), zeocin (such as the ble gene), kanamycin or G418 (such as the nptII or aphVIII genes), spectinomycin (such as the aadA gene), neomycin (such as the aphVIII gene) and paromomycin (such as the aphVIII gene) or may confer resistance to herbicides such as phosphinothricin (for instance the bialaphos resistance (bar) gene) or norflurazon (a modified phytoene desaturase gene).

The selectable marker gene is preferably a codon-optimised positive selectable marker gene, optimised for expression in an algal or microalgal cell into which it will be inserted.

A positive selectable marker gene allows the determination of whether or not the integration cassette has been inserted; if the integration cassette has been inserted, the positive selectable marker gene will be expressed, imparting antibiotic or herbicide resistance to the algal cell. Subjecting the cells to selective treatment will mean that only cells which have the integration cassette successfully inserted therein will survive. In some embodiments, in particular the promoter trap and the combined promoter/polyA trap, the positive selectable marker gene may be used to indicate that the integration cassette has been successfully inserted within an actively expressed gene.

In one embodiment, the positive selectable marker gene may be fused-in-frame to an enhanced green fluorescent protein coding sequence, variants thereof or other sequences encoding a fluorescent tag. In this embodiment, the positive signal shown by initial antibiotic or herbicide resistance is confirmed by a fluorescent marker. In an embodiment where the site-specific recombinase sequences are compatible, upon application of a site-specific recombinase, cells which have successfully excised the positive selectable marker gene may be enriched by flow cytometry to identify those cells within a given transformed population of cells that have specifically lost the fluorescent signal attributable to the ongoing expression of the marker-fluorescent tag fusion protein in those algal cells.

Within the method of the present invention, the integration cassette may further comprise a negative selectable marker gene. A negative selectable marker gene is a gene whose expression imparts sensitivity to a compound to the host cell. The use of a negative selectable marker gene allows for an excision of a region of the integration cassette containing the negative selectable marker gene to be monitored by subjecting host cells suspected of having excised the negative selectable marker gene to the compound to which the host cells will be sensitive if the negative selectable marker gene is expressed; surviving cells have excised the negative selectable marker gene.

The negative selectable marker gene is preferably fused in-frame with the positive selectable marker gene. This will allow the negative selectable marker gene to utilise the promoter and 3′ UTR elements utilised by the positive selectable marker gene (i.e. the promoter present within the integration cassette and an endogenous 3′ UTR for the polyA trap; an endogenous promoter and the 3′ UTR present within the integration cassette for the promoter trap; an endogenous promoter and an endogenous 3′ UTR for the promoter/polyA trap). In one embodiment, the positive selectable marker gene and the negative selectable marker gene may be separated by a sequence encoding a short self-cleaving peptide known as a 2A peptide. “2A peptides” are described in more detail below, and function to allow the transcription and translation of the positive and negative selectable marker genes into a single polypeptide chain, which is subsequently cleaved into two separate peptides which function independently.

The negative selectable marker gene may be selected from the group consisting of the E. coli or fungal cytosine deaminase gene (codA; confers sensitivity to fluorocytosine), the D-amino acid oxidase gene (DAAO; depending upon the algal strain confers sensitivity to D-amino acids including D-Isoleucine and D-Valine) and the herpes simplex virus thymidine kinase gene (TK; confers sensitivity to gancyclovir).

If the negative selectable marker gene is E. coli codA, the E. coli uridyl phosphoribosyltransferase (UPP) coding sequence may be fused to the C-terminal end of codA in order to improve the efficiency of this negative selectable marker. Preferably the coding sequence of the fungal or E. coli codA gene is manipulated for enhanced expression in algal strains for example by codon optimisation.

### Site-Specific Recombination Sites:

The site-specific recombination sites present within the integration cassette described herein are short nucleic acid sequences, typically 30 to 60 base pairs in length, representing:

(a) sites that will be recognized by a site-specific recombinase;
(b) sites to which a site-specific recombinase will bind; and
(c) sites at which a site-specific recombinase will catalyse a recombination event.

The site-specific recombination sites may be sites recognised by any type of site-specific recombinase that functions within an algal cell. In particular, members of the serine recombinase family and the tyrosine recombinase family are preferred (Hirano N, Muroi T, Takahashi H, Haruki M. (2011) “Site-specific recombinases as tools for heterologous gene integration”, Appl. Microbiol. Biotechnol. 92(2):227-239). In one example embodiment, the site-specific recombination sites are sites recognised by a recombinase selected from the group consisting of:

actinophage R4 recombinase (sre) (SEQ ID NO:9 or SEQ ID NO:10), B3 recombinase of Zygosaccharomyces bisporus (SEQ ID NO:11), Flp recombinase of the yeast 2 micron plasmid (SEQ ID NO:12, SEQ ID NO:13 or SEQ ID NO:14), the bacteriophage φBT1 integrase (SEQ ID NO:15), the Streptomyces actinophage TG1 recombinase (SEQ ID NO:16), the B2 (SEQ ID NO:17), SM1 (SEQ ID NO:18), R/RS (SEQ ID NO:19), the KD1 (SEQ ID NO:20) recombinases of Zygosaccharomyces bailii, Zygosaccharomyces fermentati, Zygosaccharomyces rouxii and Kluyveromyces lactis, respectively (Esposito D, Scocca J J (1997) The integrase family of tyrosine recombinases: evolution of a conserved active site domain. Nucleic Acids Res. 25(18):3605-3614), and active variants thereof.

When R4 attB recombinase recognition sites are utilised as the two site-specific recombination sites, attP or attB sites can be used within the initial gene trapping construct; the two attB sites can be present within the integration cassette in an inverted configuration with respect to each other, such that one attB site has the sequence (SEQ ID NO:9) and the other attB site has the sequence (SEQ ID NO:10), or, alternately, in a direct configuration The inverted arrangement is depicted in FIG. 4.

In one example embodiment, the two site-specific recombination sites within the integration cassette may be the same type and as such capable of interacting with each other in the presence of the relevant site-specific recombinase. This embodiment has an advantage that one or more selectable marker genes can be introduced into the algal genome as part of an integration cassette, which may additionally contain one or more target genes, with the one or more selectable marker genes subsequently being excised from the algal genome in the presence of the relevant site-specific recombinase. Here, the site specific recombination sites are preferably recognised by a site-specific recombinase exogenous to the species of algae or microalgae to be used so that the one or more selectable marker genes are only excised following provision of the recombinase. The mechanics of this embodiment are further described below.

In another example embodiment of the present invention, the two site-specific recombination sites present within the integration cassette may be of different types, such that they are heterospecific, and, as such, cannot mutually interact. The use of two different site-specific recombination sites that are not able to interact mutually permits the integration cassette to be inserted into the algal genome and subsequent gene replacement using a target cassette, as elucidated in more detail below.

Within this example embodiment, the two site-specific recombination sites may be of distinct types that naturally interact with different recombinases. Alternatively, the two site-specific recombination sites may be of the same type, i.e. naturally interact with the same recombinase, with one of the sites containing a mutation from a wild-type sequence, such that the two site-specific recombination sites cannot interact with one another. Here, the two site-specific recombination sites may be a wild-type FRT site (SEQ ID NO:12), and a mutated FRT site, referred to as either FRT3 (SEQ ID NO:13) or FRT5 (SEQ ID NO:14) or other mutated FRT sites derived by standard experimentation. This embodiment is not limited to the recombinase sequences listed; any functioning recombinase target either naturally occurring or synthetic can be utilised to effect the methods of the invention.

As described throughout, the integration cassette must contain two site-specific recombination sites flanking the selectable marker gene. However, the integration cassette may also contain one or more (e.g. two, three, four, five, six or more) additional site-specific recombination sites, which may be of the same type as one, but preferably not both, of the other two site specific recombination sites present in the integration vector, or of a different type. The integration cassette may therefore contain, for example, a total of 2, 3, 4, 5, 6, 7 or 8 site-specific recombination sites.

Intronic Sequences Found within the Integration Cassette:

The integration cassette may further comprise one or more intronic sequences. An intronic sequence is thought to function as an expression stabilising influence on transgene expression. Its function is therefore analogous to an enhancer. The intron is also helpful in determining whether or not the target gene is expressing correctly from the algal genome as RT-PCR should amplify correctly spliced mRNAs from which any introns would be expected to be spliced out. Finally, the presence of an intron allows recombination sites to be included, without affecting the coding portions of any selectable marker genes. However, other designs where the recombination sites are not present within an intron or untranslated sequence but are included as an in-frame fusion at the beginning of the target gene coding domain, could also be optionally applied as an integration cassette.

The intronic sequence may be optionally an algal intronic sequence or a synthetic intronic sequence. An algal intron is particularly useful, because it more closely mimics the structure of a native algal gene, thereby being more likely to result in stable, long-term expression of any inserted transgene. In a preferred embodiment, the intronic sequence is intron 1 from the Chlamydomonas reinhardtii RbcS2 gene (SEQ ID NO:21).

In a particularly preferred embodiment, the intronic sequence may be located towards the beginning of the integration cassette sequence. However, additional introns, including additional copies of the RbcS2 intron 1, may be optionally included at any position within the integration cassette.

A person skilled in the art will be able to readily identify alternatives to the components of the integration cassette named above by routine experimentation using naturally occurring sequences, variants of naturally occurring sequences that may be beneficial in terms of codon bias, or in the inclusion or exclusion of restriction endonuclease recognition sites, or synthetic sequences. Those components named above are not intended to be limiting.

Mechanism of Introduction into Algal Cells:

Elements relating to a method of introducing of the integration cassette into the algal cell will now be described in more detail below.

### Integration Vector:

In one embodiment, the integration cassette may be contained within an integration vector which contains additional sequences to those forming the integration cassette. The integration vector may be a plasmid, a cosmid, a BAC, a YAC or an Agrobacterium based T-DNA plasmid. Alternately, the integration sequences could be contained within the context of an algal artificial chromosome sequence. Integration cassettes may also be optionally contained within the context of a DNA fragment, such as a restriction fragment or PCR amplified fragment or a synthetic fragment. Optionally, coding regions are optimised for expression in the algal host.

### Linearisation of Integration Vector Containing Integration Cassette:

The integration vector containing the integration cassette, or the integration cassette itself, may comprise one or more specific restriction endonuclease sites that are used to convert the circular DNA into a linearised form and optionally to isolate the integration cassette. A restriction endonuclease site is a recognition site for a restriction endonuclease; a specific nucleic acid motif at which the restriction endonuclease will cleave the integration vector or integration cassette. Cleavage of the integration vector is advantageous in converting a circular vector into a linear DNA sequence for increased efficiency of integration into the algal or microalgal cell genome. Additional one or more restriction endonuclease sites may optionally be useful to cleave vector backbone sequences from the integration vector. The one or more restriction endonuclease sites may be selected from those sites recognised by commercially available restriction endonucleases. Where an integration cassette is used, the restriction endonuclease is preferably selected from those which do not cut within the integration cassette sequence, namely preferably cutting the vector just outside the integration cassette. Preferred restriction endonuclease sites used within such a context include BamHI, NruI, PvuI, PvuII, XmnI and NotI, although this approach is not limited to these restriction endonuclease sites.

Introduction of Integration Cassette into Algal Genome:

In the method of the present invention, the integration cassette may be beneficially introduced into the algal genome as a cassette sequence, for example as a linear, double-stranded PCR generated DNA or a purified restriction fragment, or as a double-stranded synthetic fragment generated for example by annealing two synthetic oligonucleotides or as part of an integration vector sequence. Optionally, the integration cassette could be introduced into the algal genome as a self-replicating, entirely artificial algal chromosome. Regardless of whether or not additional vector sequences are present, the integration cassette will be inserted into the algal genome in the same manner.

The integration vector or integration cassette is usually, but not exclusively, linearised before it is transformed into the algal cell. Such linearisation may be performed by exposing the integration vector or the integration cassette to a restriction endonuclease capable of acting on a restriction endonuclease target site present within the integration vector or at the beginning or end of the integration cassette.

The integration vector or integration cassette may be introduced into the algal cell by any transformation method known in the art including, but not limited to electroporation, glass bead transformation, silicon carbide whiskers, biolistic transformation, or Agrobacterium tumefasciens mediated transformation.

The integration vector or integration cassette may integrate into the algal genome through non-homologous, namely random, integration, or through homologous recombination. In a homologous recombination scenario, the integration cassette may further optionally comprise one or more nucleic acid sequences homologous to part of the algal genome positioned at the 5′ or the 3′ end of the integration cassette. In a preferred embodiment the integration cassette comprises a nucleic acid sequence homologous to the algal genome positioned at both the 5′ and 3′ ends of the integration cassette. These sequences may be independently selected. The one or more nucleic acid sequences homologous to part of the algal genome may be independently 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 or more base pairs in length.

In one example embodiment, one or both of the nucleic acid sequences homologous to part of the algal genome may comprise one or more nucleic acid mutations relative to the wild-type algal genome sequence. The mutations may be additions, substitutions or deletions of one or more (e.g. 1, 2, 3, 4, 5, 6, 7, 8, 9, 10, 11, 12, 13, 14 or 15) nucleotides leading to missense or nonsense mutations or deletions or insertions of specific amino acids when these mutations are positioned within protein coding domains, or possibly exon-skipping where mutations affect intron-exon splice junctions. Following insertion of the integration vector or integration cassette into the algal genome through homologous recombination, a portion of, or the entire mutant version of the sequence homologous to the algal genome will be inserted into the algal genome in place of the wild-type algal sequence, effectively introducing a mutation into the algal genome.

### Target Gene(s):

Various mechanisms, namely methods, for use in inserting one or more target genes into the algal genome using the methods of the present invention are discussed in more detail below. Using these methods, any one or more target genes may be introduced into the algal genome. Generally, the one or more target genes will be exogenous genes, the expression of which by the algal cell is desired.

The target gene may be an open-reading frame encoding a desired polypeptide sequence, an RNAi-type knockdown sequence such as that previously described to work in Chlamydomonas (Rohr J, Sarkar N, Balenger S, Jeong B R, Cerutti H. (2004) Tandem inverted repeat system for selection of effective transgenic RNAi strains in Chlamydomonas. Plant J. 40(4):611-621), a combination sequence in which an open-reading frame sequence is followed by an RNAi sequence such that the resultant transgene would be designed to express the desired polypeptide as well as affect the reduction or silencing of expression of an endogenous algal gene.

In one embodiment, a single gene may be inserted into the algal cell using the method of the invention. Alternatively, multiple target genes (e.g. 2, 3, 4, 5 or more genes) may be inserted into the algal genome. These target genes may all be mutually different, or they may represent multiple versions of one or more genes.

Where multiple target genes are inserted into the algal genome, these may be inserted in a single step, whereby all the target genes are present within a single target cassette or integration cassette and are inserted into the algal genome together. Alternatively, the multiple target genes may be inserted into the algal genome in multiple steps, e.g. 2, 3, 4, 5 or more steps, in order to create a tandem array through gene stacking. In each step, one or more target genes may be inserted into the algal genome.

In the aforementioned embodiment where multiple target genes are inserted into the algal genome in a single step, these target genes may be organised such that the respective reading frames are fused-in-frame and are separated by sequences encoding self-cleaving peptides. Under this scenario, the sequence is transcribed and translated into a single polypeptide chain, which is subsequently cleaved into multiple (e.g. 2, 3, 4, 5 or more) peptides. These self-cleaving peptides are known as 2A peptides. “2A peptides” are short, self-cleaving peptides that are viral in origin, originally being described in picornaviruses such as the foot and mouth disease virus (FMDV), and have been shown to be functional in multiple eukaryotic cell types including plants. Self-cleaving peptides are short sequences of approximately 20 amino acids that, when placed within the context of a polypeptide sequence and expressed within a cell, result in co-translational cleavage of the expressed polypeptide. 2A peptides have been described from various viral sources and each contains the consensus motif Asp-Val/Ile-Glu-X-Asn-Pro-Gly-Pro (DV/IEXNPGP; SEQ ID NO:22) where cleavage of the 2A sequence takes place between the terminal Gly and Pro (underlined) (Donnelly M L, Hughes L E, Luke G, Mendoza H, ten Dam E, Gani D, Ryan M D. (2001) The ‘cleavage’ activities of foot-and-mouth disease virus 2A site-directed mutants and naturally occurring ‘2A-like’ sequences. J. Gen. Virol. 82:1027-1041; de Felipe P, Luke G A, Hughes L E, Gani D, Halpin C, Ryan M D. (2006) E unum pluribus: multiple proteins from a self-processing polyprotein. Trends Biotechnol 24:68-75).

2A peptides can be used within the context of the present invention to express effectively two or more (e.g. 3, 4, 5, 6, 7, 8, 9, 10, 11, 12, 13, 14, 15, 16, 17, 18, 19, 20 or more) functional proteins from a single mRNA (Tang W, Ehrlich I, Wolff S B E, Michalski A M, Wolfl S, Hasan M T, Luthi A, Sprengel R (2009) Faithful expression of multiple proteins via 2A-peptide self-processing: A versatile and reliable method for manipulating brain circuits. J. Neurosci. 29(27):8621-8629; Kim J H, Lee S- R, Li L- H, Parl H- J, Park J- H, Lee K Y, Kim M- K, Shin B A, Choi S- Y. (2011) High cleavage efficiency of a 2A peptide derived from porcine teschovirus-1 in human cell lines, zebrafish and mice. PLoS One 6(4):e18556). Unlike fusion proteins, where fusion partners generally remain fused and must function within the context of the full fusion protein, polypeptides that are designed to include 2A self-cleaving peptides between fusion protein partners, result in mostly (dependent upon the 2A sequence used and the cell type) cleaved proteins, where the fusion partners are able to function independently, including trafficking to specific cellular compartments (Kim J H, Lee S- R, Li L- H, Parl H- J, Park J- H, Lee K Y, Kim M- K, Shin B A, Choi S- Y. (2011) High cleavage efficiency of a 2A peptide derived from porcine teschovirus-1 in human cell lines, zebrafish and mice. PLoS One 6(4):e18556).

Within this embodiment, the target genes joined by the self-cleaving peptides may be any type of target genes, including all the potential target genes discussed above.

Here, self-cleaving peptides may be employed in three contexts with regard to algal genome modification. Firstly, a sequence encoding a 2A peptide, such as the Thoseaasigna virus 2A (T2A) peptide (EGRGSLLTCGDVEENPGP; SEQ ID NO:23) or other sequences encoding 2A peptides, may be placed between sequences encoding for a positive selectable marker, such as hygro or ble or nptII and a negative selectable marker, such as codA and/or UPP. Secondly, a sequence encoding a 2A peptide may be placed such that a 2A peptide coding sequence is positioned at the beginning of a first target gene coding sequence. Such an arrangement results in cleavage of the encoded polypeptide in question, away from any N-terminal polypeptide sequences that might be expressed from the endogenous native trapped gene or transgene. Thirdly, a sequence encoding 2A peptides may be used between multiple target genes such that polypeptides that are encoded by any gene that is designed to be inserted into trapped sites or transgenes are fused-in-frame, immediately downstream of the 2A peptide but are liberated through the co-translational cleavage of the 2A peptide sequences.

Other 2A peptides optionally function within a similar context in algal transgenesis and a person skilled in the art could readily, using the invention, create variants of the 2A peptide that would work in this embodiment. Variants optionally include entirely synthetic 2A peptides, conforming to the consensus sequence. The present invention includes the optional use of all 2A peptide variants.

### Mechanisms of Insertion, Deletion and Monitoring:

Various different mechanisms, namely methods, of effecting insertion of the integration cassette into the algal genome, insertion of one or more target genes into the algal genome, and excision of the one or more selectable marker genes will next be described in more detail.

A result of the initial transformation is the derivation of a library of “primed” algal strains with a selectable marker gene and site-specific recombination sites inserted at unique and defined sites within the algal nuclear genome. Depending upon the transformation efficiency of the algal parent strain, several to hundreds of primed strains may form the initial library of “primed” algal strains, namely library of integrants. The integrants are categorised based upon the site of insertion within the genome and the relative expression level of the selectable marker, based upon quantitative or semi-quantitative RT-PCR.

The library of integrant strains can also be categorised based on a trait of the parent algal cell to understand better the parent strain. For example if the integration fragment disrupts the expression of a native gene then a better understanding of the role of that gene may result. In particular if the interest in the strain is in the production of a metabolite, then integrant strains may also be assessed for production of that metabolite. Strains may be ranked and this may lead to a preferred strain to go forward for gene replacement eg one where the desired metabolite is highly produced. Additionally, integrant strains can be analysed to increase understanding of the strain and its utility as a producer of that metabolite. For example strains that produce increased amounts of the target may harbour a gene disruption that improves production of the desired metabolite. One of skill in the art will appreciate there are numerous ways in which this can occur, for example but not limited to one of the following; a negative regulator, a competitor for substrate, a competitor for a positive regulator. A strains that is selected for its ability to be selected for on the positive selection compound and further has an increased production of a target metabolite may represent a strain to take forward for deletion of the inserted cassette if compatible recombinase sites are present, or for integration to replace the selectable marker with a target gene, Alternatively, an integrant strain may produce the desired metabolite in a reduced quantity. This is generally not preferred but the strain may be analysed genetically to elucidate the gene function relationship that has been impacted. Alternatively, metabolite production may be assessed and the production of an undesired metabolite may be monitored. Reduction in the production of an undesired metabolite would be beneficial. An undesired metabolite may compete with the desired metabolite for available substrates, regulating elements or co-purify with the desired metabolite.

The primed algal cells may be further modified by effecting gene replacement using a target cassette encoding one or more target genes or gene stacking of target genes into the trapped site to build up a tandem gene array. These methods are advantageous over the methods of the prior art because they allow one or more target genes to be inserted into the algal genomes at defined locations, permitting comparisons between identical target genes inserted at different locations or different target genes inserted at the same position within a given algal genome. In each case providing a predictable and directed genetic outcome based on the primed cell.

### Recombinase-Mediated Replacement:

Algal cells that have been primed by the insertion of an integration cassette may be used for the subsequent insertion of a target gene in place of the selectable marker gene.

In this example embodiment, the two site-specific recombination sites present on the inserted integration cassette are a type I and a type II site-specific recombination site, which are heterospecific, and as such are not capable of interacting and excising the selectable marker gene posited therebetween.

In this embodiment, the method further comprises the steps of providing a target cassette comprising a sequence of one or more target genes sequences flanked by a type I site-specific recombination site and a type II site-specific recombination site, which are mutually different but correspond to the type I and type II site-specific recombination sites present within the inserted integration cassette; the targeted site-specific recombinase mediated insertion of the target cassette into the algal genome is achieved by effecting recombination between corresponding type I and type II site-specific recombination sites flanking the target gene sequence and located in the algal genome, such that the target gene sequence is introduced into the algal genome, replacing the selectable marker gene in a predictable manner.

The insertion of the target cassette into the algal genome at the primed site ensures that the target gene will be positioned at a defined location, and preferably within an actively expressed gene so that it will be actively expressed by the modified algal cell. If the target gene represents an open-reading frame encoding a desired polypeptide sequence, the target cassette should be inserted into the primed site within the algal genome such that upon successful recombinase-mediated replacement, the reading-frame of the target would be in the correct frame with the promoter and 3′ UTR which have either been inserted during the priming step from the integration cassette, or have been harnessed from the algal cell.

The target cassette may further include the end of an intron including an intron splice acceptor and/or a consensus splice donor sequence. Inclusion of these sequences effectively converts the target cassette into what amounts to be an exon within the newly engineered gene. When the target gene sequence is flanked at its 5′ end by an intron splice acceptor and at its 3′ end by an intron splice donor sequence, these sequences are recognised within the context of the newly created gene and used as splice sites to correctly splice the target gene sequence into the endogenous or transgene sequences. This is important as the relative position of intron splice sites will determine the ultimate reading frame of the spliced sequence as it is spliced into the endogenous or transgene sequences to create the mRNA.

Insertion of a target cassette into a primed algal genome may be performed multiple times in order to insert multiple genes into the algal genome in a tandem array through a process of gene stacking. In this embodiment, insertion of a target cassette into the primed algal genome may occur 2, 3, 4, 5 or more times. Each time, the one or more target genes within the target cassette may be the same or different.

In the preferred embodiment, the insertion of the target cassette into the primed algal cell is monitored using a negative selectable marker gene present within the algal cell following insertion of the integration plasmid. Site-specific recombinase mediated insertion of the target cassette into the primed algal cell will necessarily result in excision of the negative selectable marker gene, along with the fused positive selectable marker if one is present. Exposure of the modified algal cell to the compound to which cells expressing the negative selectable marker gene are sensitive (e.g. fluorocytosine when codA is the negative selectable marker) will kill all cells that still express the negative selectable marker gene, and will leave only those cells that have correctly inserted the target cassette in place of the negative selectable marker gene surviving. Recombinase-mediated replacement is illustrated in FIGS. 2, 3 and 4.

### Simultaneous Insertion of Integration Cassette and Deletion of Endogenous Gene:

In one embodiment of the invention, the integration cassette may be inserted into the algal genome with the simultaneous deletion of an endogenous algal gene or part of an endogenous algal gene. In this embodiment, the integration cassette is incorporated into the algal genome through homologous recombination such that an endogenous algal gene, or part thereof, is effectively replaced by the integration cassette.

The success of this method and the extent of the deletion will be dependent upon the location of insertion of the integration cassette, which can be directed with the help of additional nucleotide sequences at the 5′ and 3′ ends of the integration cassette. Utilising two nucleotide sequences which are homologous to the algal genomic sequences directly flanking the endogenous algal gene that is to be replaced/disrupted will result in simultaneous excision of the endogenous algal gene, or portions thereof, and insertion of the integration cassette, i.e. directed replacement of a portion of an endogenous algal gene with the integration cassette, as depicted in FIG. 6.

Within this embodiment, the integration cassette may contain one or more target genes which are inserted into the algal genome as part of the integration cassette. As elucidated in the foregoing, any target genes may be optionally employed, including single target genes, multiple target genes, and multiple target genes separated by sequences encoding self-cleaving peptides. Any such target genes are preferably positioned at the 5′ or 3′ end of the integration cassette such that subsequent site-specific recombinase-dependent excision of the one or more selectable marker genes will leave the one or more target genes within the algal genome.

In this embodiment, the two site-specific recombination sites flanking the one or more selectable marker genes are preferably of a mutually similar type such that they can interact, excising the one or more selectable marker genes, upon effecting site-specific recombination, as described in more detail below. Here, the site-specific recombination sites used are preferably recognised by a recombinase exogenous to the algae or microalgae so that the one or more selectable marker genes are not excised from the integration cassette as soon as it is introduced into the algal or microalgal cell.

Within the homologous recombination scenario, a key advance provided by the invention is an ability to remove the one or more selectable marker genes in a recombinase-dependent manner. By removing the one or more selectable marker genes, leaving behind a single recombination site, as illustrated in FIG. 6, and, in some instances a subtle mutation within the targeted gene, the modified strains more closely resemble strains containing a gene modified through a mutagenesis approach, effectively redefining such strains as ‘non-GM’ under certain definitions of the term as they will have a modification of an endogenous gene only and will express no foreign genetic material.

Determining Whether or not the Integration Cassette has been Incorporated:

Following insertion of the integration vector or integration cassette into the algal genome, the status of the incorporated integration cassette can be determined. The purpose of such determination is to identify those algal cells within which the integration cassette has inserted itself, permitting one or more subsequently inserted target genes to be expressed.

The relative location of the integration cassette is preferably determined using the positive selectable marker gene, wherein expression of the positive selectable marker gene is indicative of the positioning of the integration cassette within an algal gene. In some embodiments, particularly in embodiments including the promoter trap and the combined promoter/polyA trap, it may be desirable to determine whether or not the integration cassette has been inserted within an actively expressed gene. Herein, the term “actively expressed” denotes that expression of the selectable marker gene is detectable.

In a preferred embodiment, the positive selectable marker gene is an antibiotic or herbicide resistance gene, and the insertion of the integration cassette is determined by applying the relevant antibiotic or herbicide to the transformed cells. The relevant antibiotic may be selected from the group consisting of hygromycin B, zeocin, kanamycin, G418, neomycin, paromomycin, and spectinomycin, and the relevant herbicide may be selected from the group consisting of compounds such as phosphinothricin and norflurazon.

Only if the integration cassette has been inserted into the algal genome within an expressed gene will an antibiotic or herbicide resistant colony appear; each colony theoretically represents a separate gene trap event where an endogenous algal gene has been trapped and its control elements harnessed to drive expression of the positive selectable marker gene. Initial resistant strains are ideally restreaked on fresh selective media plates to confirm their resistance to the antibiotic or herbicide in question, prior to further validation.

### Determining the Location of Integration:

Once it has been determined that the integration cassette has been inserted into the algal genome, the actual location of the insertion may be determined. The method of the present invention may therefore comprise a further step of determining the position of the integration cassette within the algal genome. Any known method of position determination may be used to determine the position of the integration cassette within the algal genome. In those algal strains for which the nuclear genome has been sequenced, the position of the integration cassette can be determined by a PCR-based genomic walking approach using the integration cassette sequence as the starting point to obtain flanking DNA sequences. Alternately, 3′RACE (rapid amplification of cDNA ends) or 5′RACE may be used to identify the respective insertion sites for the polyA trap integration cassette (3′RACE), promoter/polyA trap integration cassette (3′ and 5′RACE) and promoter trap integration cassette (5′RACE). Individual algal colonies are expanded and validated for site of insertion, identification and exclusion of any integrant strains with more than one insertion site and properties such as relative growth rate under defined conditions.

In those algal strains for which only limited genomic and/or cDNA sequence is available, a determination of insertion site may be optionally made based upon nucleic acid alignments against known algal genomic and cDNA sequences, including sequences that are publicly available for other algal strains with inferences drawn based upon relative percentage of homology/similarity of the trapped sequences to known algal genes.

One skilled in the art will appreciate that it is not a requirement to determine the position of integration to determine that a strain is useful.

Introducing Target Gene Sequence into Primed Algal Cell:

The present invention also includes a method for introducing a target gene sequence into a primed algal cell, wherein the method comprises steps of
(a) providing a primed algal cell comprising an integration cassette comprising a type I site-specific recombination site and a type II site-specific recombination site flanking a selectable marker gene, wherein the type I site-specific recombination site is different from the type II site-specific recombination site such that it is heterospecific and as such cannot interact with the type H site-specific recombination site, within the algal cell genome;
(b) providing a target cassette comprising a target gene sequence flanked by a type I site-specific recombination site and a type II site-specific recombination site; and
(c) effecting targeted site-specific recombinase-mediated insertion of the target cassette into the algal genome by effecting recombination between corresponding type I and type II site-specific recombination sites flanking the target gene sequence and located in the algal genome, such that the target gene sequence is introduced into the algal genome.

It will be appreciated that this method corresponds to performing the method described above in an algal cell that has already been primed. Herein, the term “primed” indicates that an algal cell contains within its nuclear genome an integration cassette, namely two site-specific recombination sites flanking at least one selectable marker gene.

In some embodiments, particularly in respect of the promoter trap and combined promoter/polyA trap, it is preferable for the primed algal cell to contain the integration cassette within an actively expressed algal gene. Herein, the term “actively expressed” denotes that expression of the selectable marker gene is detectable.

### Effecting Site-Specific Recombination:

As elucidated above, the methods of the present invention may require recombination to be effected between two corresponding site-specific recombination sites. Site-specific recombination may be effected by any method known in the art. In particular, site-specific recombination may be effected by providing a relevant site-specific recombinase to the algal cell or providing a DNA sequence encoding a relevant site-specific recombinase to the algal cell. As used herein, the term “relevant site-specific recombinase” is used to refer to a site-specific recombinase capable of acting upon the site-specific recombination sites within the algal genome and/or a target cassette(s) present within the algal cell. In preferred embodiments the recombinase may be R4 (sre) recombinase (SEQ ID NO:24), B3 recombinase (SEQ ID NO:25), Flp recombinase (SEQ ID NO:26), βBT1 recombinase/integrase (SEQ ID NO:27), TG1 recombinase (SEQ ID NO:28), B2 recombinase (SEQ ID NO:29), SM1 recombinase (SEQ ID NO:30), R/RS recombinase (SEQ ID NO:31), KD1 recombinase (SEQ ID NO:32), and active variants thereof. Cre recombinase and phiC31 integrase and variants thereof could also be applied within this context. The sequence of the site-specific recombinase may also be codon-optimised for expression within the particular algal strain.

In a preferred embodiment, site-specific recombination is affected by providing a DNA sequence encoding a relevant site-specific recombinase to the algal cell. This recombinase may be encoded on a plasmid or on a linear DNA fragment generated from a plasmid or through PCR amplification. In embodiments where a target cassette is used, the site-specific recombinase and the target cassette may be present on the same plasmid. The site-specific recombinase will be functionally expressed in the algal cell. The site-specific recombinase may be under the control of a strong algal or microalgal promoter such as the Hsp70A, RbcS2 tandem combination, or a heterologous promoter. An algal or microalgal intron sequence, such as the RbcS2 intron 1 sequence, and a microalgal 3′UTR such as the RbcS2 or beta-2-tubulin 3′UTR may also be provided in functional connection with the site-specific recombination gene. The recombinase may also be introduced into the cell as an mRNA molecule or as a recombinant protein, including a cell permeant recombinase polypeptide that is expressed and secreted by an algal cell; see United States patent application US2003/0027335.

### Cassettes and Cells

### Integration and Target Cassettes:

Included within the scope of the present invention are the integration and target cassettes used for any of the methods described here.

### Algal Cells:

Algal cells used in the methods of the present invention are preferably microalgal cells although it is considered likely that the approach will work equally well in macroalgal cells. The microalgal cells are preferably Chlamydomonas reinhardtii strains, Chlorella species including Chlorella vulgaris, Chlorella sorokiniana and Chlorella (Auxenochlorella) protothecoides, Dunaliella salina, Haematococcus pluvialis, Ostreococcus tauri, Nannochloropsis species, and Scenedesmus species. Other microalgal cells include diatoms such as Phaeodactylum tricornutum.

Included within the scope of the present invention are primed algal cells for use in a method of introducing a target gene sequence into a primed algal cell and modified algal cells produced by any of the methods of the invention.

### BRIEF DESCRIPTION OF DRAWINGS

Embodiments of the present invention will be described, by way of example only, with reference to the following diagrams, wherein:

FIG. 1 is an illustration of integration cassettes for use in:

A) a polyA trap;

B) a promoter trap; and

C) a promoter/polyA trap. A text legend shown in FIG. 1 also applies to FIGS. 2 to 8 and 16.

FIG. 2 is an illustration of a polyA trap followed by a gene replacement:

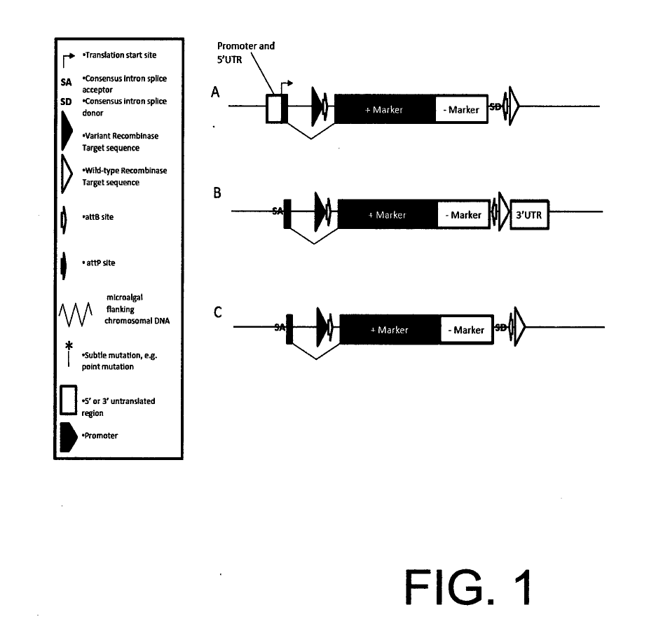

A) An algal cell is transformed with a linear DNA fragment containing the integration cassette which may have been generated from linearised plasmid DNA or from a purified linear construct DNA (free of flanking vector backbone) or synthetically;

B) The trapped site in the algal genome is shown; cells which have integrated the construct are selected for using the positive marker;

C) The sequence integration plasmid is co-transformed with a recombinase plasmid or with a recombinase mRNA or polypeptide, permitting transient expression of a site-specific recombinase; recombinase-mediated cassette exchange occurs, and is selected for based on loss of the negative marker;

D) The target gene is inserted at defined genomic site and expressed as a fused mRNA with endogenous algal 3′UTR sequences.

FIG. 3 is an illustration of a promoter gene trap followed by gene replacement:

A) The algal cell is transformed with a linear DNA fragment containing the integration cassette which may have been generated from linearised plasmid DNA or from a purified linear construct DNA (free of flanking vector backbone) or synthetically;

B) The trapped site in the algal genome is shown; cells which have integrated the construct are selected for using the positive marker;

C) The sequence integration plasmid is co-transformed with a recombinase plasmid, permitting transient expression of a site-specific recombinase; recombinase-mediated cassette exchange occurs and is selected for based on loss of the negative marker;

D) The target gene is inserted at defined genomic site and expressed as a fused mRNA with an endogenous algal promoter and transcription start site.

FIG. 4 is an illustration of a promoter/polyA gene trap followed by gene replacement:

A) The algal cell is transformed with a linear DNA fragment containing the integration cassette which may have been generated from linearised plasmid DNA or from a purified linear construct DNA (free of flanking vector backbone) or synthetically;

B) The trapped site in the algal genome is shown; cells which have integrated the construct are selected for using the positive marker;

C) The sequence integration plasmid is co-transformed with a recombinase plasmid containing a target gene, permitting transient expression of a site-specific recombinase; recombinase-mediated cassette exchange occurs and is selected for based on loss of the negative marker;

D) The target gene is inserted into a defined genomic site and expressed as a fused mRNA under control of an endogenous algal promoter and transcription start site and flanked by an endogenous 3′UTR sequence.

FIG. 5 is an illustration of a simultaneous insertion of integration cassette and target gene followed by selectable marker gene removal:

A) An algal transgenic gene expression and marker removal vector, containing a target gene, is transformed into the algal cell in linearised plasmid DNA or purified, linear construct DNA (free of flanking vector backbone) or synthetic form;

B) The trapped site in the algal genome is shown; cells which have integrated the construct are selected for using the positive marker and cells are screened for high expressing integrant strains and to determine transgene copy number;

C) The transformed algal cell is further transformed with a recombinase plasmid, permitting transient expression of a site-specific recombinase; deletion of the marker genes is affected and is selected for based on loss of the negative marker as recombined integrant strains lose positive-negative marker cassette; integrant strains are screened for expression of target gene. Integrant strains retain recombinase targets for subsequent recombinase mediated cassette exchange or gene stacking approaches.

FIG. 6 is an illustration of a simultaneous insertion of integration cassette and partial deletion of endogenous gene followed by selectable marker gene removal:

A) there is shown an example of linearised microalgal gene targeting vector as compared to a wild-type algal gene; the algal cell is transformed with linearised plasmid DNA or purified linear construct DNA (free of flanking vector backbone); homologous recombinants are identified by screening using the positive selectable marker gene;

B) The sequence integration plasmid is co-transformed with a recombinase plasmid, permitting transient expression of a site-specific recombinase; recombinase mediated deletion of the marker genes; deletion of the marker genes is effected and is selected for based on loss of the negative marker as recombined integrant strains lose positive-negative marker cassette;

C) Marker-free algal gene knockout is shown.

FIG. 7 is an illustration of an incorporation of gene mutation and the selectable marker gene removal:

A) there is shown an example of linearised algal gene targeting vector with a mutation relative to the corresponding wild-type algal gene; the algal cell is transformed with linearised plasmid DNA or purified linear construct DNA (free of flanking vector backbone); homologous recombinants are identified by screening using the positive selectable marker gene;

B) The sequence integration plasmid is co-transformed with a recombinase plasmid, permitting transient expression of a site-specific recombinase; recombinase mediated deletion of the marker genes; deletion of the marker genes is effected and is selected for based on loss of the negative marker as recombined integrant strains lose positive-negative marker cassette;

C) Marker-free algal gene is shown with subtle mutation.

FIG. 8 is an illustration of a promoter/polyA gene trap followed by gene replacement/2A peptide scenario:

A) The algal transgenic gene expression and marker removal vector, containing the positive and negative selectable marker genes linked by a 2A peptide, is transformed into the algal cell in linearised plasmid DNA or purified, linear construct DNA (free of flanking vector backbone) form;

B) The trapped site in the algal genome is shown; cells which have integrated the construct are selected for using the positive marker;

C) The sequence integration plasmid is co-transformed with a recombinase plasmid which also contains a target gene linked to a 2A peptide, permitting transient expression of a site-specific recombinase; recombinase-mediated cassette exchange occurs and is selected for based on loss of the negative marker;

D) The target gene is inserted into a defined genomic site and expressed as a fused mRNA under control of an endogenous algal promoter and transcription start site and flanked by an endogenous 3′UTR sequence.

FIG. 9 is an illustration of a microalgal promoter/polyA gene trap in Chlamydomonas reinhardtii:

A) Nested 3′RACE (asterisked bands sequenced and ID's shown in Table 1 below);

B) Transgene internal RT-PCR control.

FIG. 10 is an illustration of results obtained from inverse PCR based promoter walking analysis of promoter trap integrant strains produced in Chlamydomonas reinhardtii. PCR products obtained from 11 independent integrants were produced using the inverse PCR based promoter walking strategy described in Example 3. Dominant PCR products were gel-extracted and subjected to direct automated sequencing. Negative controls (−ve) represent parental CC849 cells control (−veA) and water control (−veB), respectively.

FIG. 11 is an illustration of the growth of Chlamydomonas reinhardtii gene trap integrant strains which are transgenic for a ble-codA fusion transgene. Five microliters of liquid cultures derived from three different gene trap integrant strains transgenic for the ble-codA transgene were spotted onto TAP-agar plates which were devoid of selective agent (A), 10 μg/ml zeocin (B) or 1 mg/ml fluorocytosine (FC) (C) or 50 μg/ml hygromycin B (negative control) and grown under constant illumination and a temperature in a range of 26° C. to 28° C. for a period in a range of 7 to 10 days before scoring. Gene trap integrant strains grew under conditions devoid of selective pressure was made or under conditions wherein 10 μg/ml zeocin was included in the culture medium, whereas the same integrant strains failed to grow under selection with either 1,000 μg/ml fluorocytosine or 50 μg/ml Hygromycin B. Algal strains including: Chlamydomonas reinhardtii (not shown), Chlorella species including Chlorella vulgaris (E), Chlorella sorokiniana, Chlorella (F) (now Auxenochlorella) protothecoides (not shown), Haematococcus pluvialis (G), Nannochloropsis oculata (not shown) and oceanic (not shown) and Ostreococcus tauri failed to grow in the presence of 5-fluorouracil, which represents the conversion product that is produced by the action of cytosine deaminase on fluorocytosine. Thus, it is strongly suggested that expression of transgenes possessing cytosine deaminase activity represents a general purpose negative selection strategy in microalgal strains, for example as employed in embodiments of the present invention.

FIG. 12 is an illustration of results obtained when implementing the present invention, where:

i) There is shown a table of results obtained from transforming two Nannochloropsis strains with hygro gene trap constructs. Frequencies of transformants are indicated per microgram (μg) DNA for each strain and for each construct: TUB2 indicates promoter trap, whereas SD indicates combined promoter/polyA trap. Frequencies are shown in comparison to those obtained for the VCP1_ble transgene conferring zeo resistance.

ii) There is shown a typical result of hygromycin B resistant gene trap integrant strains obtained in Nannochloropsis strains (oceanica CCAP 849/10) transformed with promoter and promoter/polyA trap constructs. Hygromycin resistant integrant strains were restreaked onto fresh plates for confirmation, and were analysed for transgene copy number and insertion site using methods described for Chlamydomonas.

FIG. 13 is an illustration of:

A) Typical results obtained from Agrobacterium-mediated transformation of a representative Chlorella vulgaris (UK native strain 4TC3/16) strain using the hygromycin promoter trap and promoter/polyA trap scenarios. Similar results were obtained for gene trap scenarios, wherein the nptII (G418 resistance) selectable marker was employed in Chlorella species including vulgaris and sorokiniana.

B) Genomic DNA PCR confirmation of the presence of the gene trap DNAs in transformed Chlorella vulgaris and sorokiniana integrant strains. Agarose gel samples were as follows: M, 2 log DNA ladder; Lanes 1 and 2, Chlorella vulgaris 4TC3/16 promoter trap integrant strains; Lanes 3 and 4, Chlorella sorokiniana UTEX 1230 promoter trap integrant strains; Lane 5, confirmed Chlamydomonas reinhardtii CC849 promoter trap clone (positive control); Lanes 6 and 7, negative controls, untransformed, parental strain genomic DNAs; Lane 8, promoter trap plasmid (positive control); Lane 9, water (PCR negative control).

FIG. 14 is an illustration of antibiotic resistant Haematococcus pluvialis cells growing after Agrobacterium-mediated co-cultivation with gene trap containing Agrobacterium strains. In this example, the transformation frequency was sufficiently high that individual colonies were not discernible. Resistant cells were successfully subcultured into liquid culture containing antibiotics at twice the concentration used to select in the plates.

FIG. 15 is an illustration of typical results obtained from biolistic transformation of Phaeodactylum tricornutum strain using a promoter and polyA trap scenario employing ble as the selectable marker. Similar results were obtained using a promoter trap construct. Colony growth is shown after 8 days of selection on zeocin containing plates with a negative control plate for reference.

FIG. 16 is an illustration of results obtained for recombinase, in this example Flp recombinase, mediated gene replacement and deletion of selectable markers in Chlamydomonas reinhardtii. In these examples: zeoS (=sensitive to, i.e. killed by, zeocin); zeoR (resistant to zeocin); hygroS (sensitive to, i.e. killed by, hygromycin B); hygroR (resistant to hygromycin B); Arrowheads represent the respective positions of diagnostic PCR primers used to screen DNAs derived from resistant cells obtained through each experimental step.

A) a ble expressing transgene (conferring resistance to zeocin) was converted to a hygro expressing transgene (conferring resistance to hygromycin B) by transient expression of Flp recombinase by electroporation of ble resistant cells with a linearised expression construct consisting of an Hsp70A-RbcS2-FlpNLS (ORF)-RbcS2 3′UTR arrangement. H1 and H2 represent two independent Hygromycin resistant strains derived from an original zeo resistant strain;

B) Subsequent hygro expressing Chlamydomonas reinhardtii strains were converted back to ble expressing strains by transformation of the recombinase plasmid in addition to a linear fragment flanked by heterospecific Flp recombinase target sequences (FRT3 and FRT WT in this instance). In this example, restoration of zeo resistance and concomitant loss of hygromycin B resistance indicated recombinase-mediated replacement of the hygro-3′UTR sequence for the ble-3′UTR resistance cassette. H1Z1 and H2Z1 represent two independent zeo resistant strains derived from two original hygro resistant strains (H1 and H2).

The invention will now be described by reference to specific examples. It should be noted that these examples are intended only to be exemplary, and are not limiting upon the scope of the disclosure.

### DETAILED DESCRIPTION OF EXAMPLE EMBODIMENTS

### Example 1

### Microalgal Promoter/polyA Gene Trap in Chlamydomonas reinhardtii

A microalgal promoter/polyA trap was performed in. Chlamydomonas reinhardtii in accordance with a scheme as shown in FIG. 4. Chlamydomonas reinhardtii strain CC849 was grown in 1 liter flasks in TAP medium with 5% CO₂/95% N₂ bubbled into the culture medium at a rate in a range of 5 to 10 ml/min at a temperature of 28° C. on an orbital platform shaker at a rotation rate of 100 rpm under constant LED lighting (66%/34% mix of red to blue) at 150 μmol/m²/s until the cultures reached a culture density in a range of 1×10⁶/ml to 1.5×10⁶/ml. Cells were pelleted by an addition of 10% Tween-20 at a dilution of 1/2000 vol/vol and centrifugation at 3,000×g for a period of 15 minutes. Cells were resuspended in TAP sucrose (40 mM sucrose) to a cell density of 4×10⁸/ml. Optionally, sheared salmon sperm DNA was added to cell suspensions to a final concentration of 200 μg/ml. Moreover, 400 μl aliquots of cell suspension were added into tubes containing 4 μg linearised, purified plasmid DNA representing the hygro promoter/polyA trap construct. Prior to electroporation, plasmid DNAs were subjected to restriction endonuclease digestion with PvuI, which cuts on either side of the integration cassette, namely approximately 160 base pairs from the 5′ end and 1.7 kilobase pairs from the 3′ end, respectively or BamHI, which cuts immediately outside the integration cassette on both the 5′ and 3′ ends. Digested plasmid DNAs were purified by phenol:chloroform:isoamyl alcohol (25:24:1 ratio) extraction and ethanol precipitation using standard molecular biology procedures prior to resuspending in nanopure water and absorbance measurement at 260 nm and 280 nm to determine their concentration and their relative purity. Cell/DNA mixes were added into electroporation cuvettes (2 mm gap) and electroporated in an electric field of 2.25 kV/cm at 25 microFarads, without added shunt resistance.

Cell suspensions were transferred into 10 ml TAP medium in 15 ml culture tubes and cultured in low light at a temperature of 28° C. for a period of 18 to 24 hours prior to plating onto 9 cm diameter TAP-agar (1.5% w/vol agar) plates containing 50 μg/ml hygromycin B in addition to 100 μg/ml carbenicillin (TAP-agar H50). Cells were pelleted gently at 1000×g for a period of 5 minutes, whereafter each pellet was gently mixed with 1 ml of a 20% (w/vol) corn starch suspension prepared in TAP sucrose and the suspension pipetted into the centre of the plate and distributed over the surface of the selective plates by gentle tilting. Plates were allowed to air dry before they were sealed with parafilm and placed under a constant illumination in a range of 150 to 200 μmol/m²/s at a temperature of 28° C. Colonies, representing, gene trap integrant strains, appeared between 5 to 14 days later, and were restreaked onto sectored TAP-agar H50 plates to confirm their resistance to hygromycin B. Ten milliliter liquid cultures were established for a selection of integrant strains. Liquid cultures were supplemented with 50 μg/ml hygromycin B and grown in racks on a shaking orbital platform mixer at a rotation rate of 100 rpm under the light and temperature conditions as described above for liquid cultures.

Thirty to fifty hygromycin B resistant colonies were obtained from each electroporation in a typical implementation of the present invention. Twenty colonies were selected for full characterisation/validation. Total RNA was extracted from 5 ml of well grown culture using conditions recommended by the manufacturer (Agilent, Absolutely RNA miniprep kit). Nested 3′RACE reactions were performed as follows:

Reverse-transcriptase reactions employed MMuLV-RT and 1 μg total RNA from each algal strain using conditions recommended by the manufacturer (New England Biolabs) with an oligo-dT adaptor primer mix (SEQ ID NO:33).

at a final working concentration of 1 μM. Five microliters of the initial RT reaction were used in two separate ‘first-round’ PCR reactions using:

1) an anchor primer (AP-OUT) combined with a transgene-specific primer; and
2) two gene specific primers flanking the modified RbcS2 intron that was included within the integration vector (this PCR reaction served as a positive control to confirm expression of the transgene as well as correct splicing of the transgene mRNA and detection of any genomic DNA contamination within the RNA samples). PCR reactions used Taq polymerase and standard 1× Taq polymerase buffer (10 mM Tris-HCl, 50 mM KCl, 1.5 mM MgCl₂, pH 8.3) supplemented with dNTP mix at a final concentration of 0.2 mM and primers at 0.2 μM in a total volume of 50 μl. PCR proceeded through an initial denaturation step of 94° C. for 1 minute, followed by 40 cycles of 94° C. 30 seconds and 68° C. 2 minutes and a final extension step of 68° C. for 10 minutes. Two microliters of the first round PCR reaction that used the AP-OUT and transgene-specific primer were used as a template for a second round of PCR (nested PCR) using identical reaction conditions except 30 cycles only were used and a nested anchor primer (AP-IN) and transgene-specific primer were used.

Ten microliter samples of each PCR reaction were analysed using agarose gel electrophoresis; see FIG. 9. 3′RACE PCR products were gel-purified using standard procedures and sequenced using automated DNA sequencing with transgene-specific primer. Sequences were screened against GenBank and Phytozome using BLAST (Basic Local Alignment Search Tool) in each instance to identify the sequences flanking the integration site at the 3′ end as well as the chromosomal site of integration. Those integrant strains for which 3′RACE PCR reactions did not yield a defined PCR product were repeated and also subjected to PCR using Deep Vent polymerase or run under conditions with 3 mM MgCl₂ and ThermoPol buffer (New England Biolabs). On average at least 50% of these reactions were successful.

In addition to validating insertion sites by 3′RACE, integrant strains were ranked for their relative abilities to grow on selective medium containing increasing concentrations of hygromycin B. Five microliters of each liquid culture was successively spotted onto gridded TAP-agar plates containing 0, 10, 50, 100, 250, 500 and 1,000 μg/ml hygromycin B, respectively. Integrant strains were scored for growth at 4 days and at 7 days.

A panel of modified strains representing unique gene trap events were maintained under selective conditions (50 μg/ml hygromycin B) in 10 ml liquid media cultures, but were also maintained as parallel cultures where no selective pressure was maintained. This was done in order to assess the relative genetic stability of the gene trapped strains in the absence of selective pressure. Cultures were routinely subcultured on a monthly schedule and restreaked onto a selection series of agar culture plates containing hygromycin B concentrations ranging from 0 μg/ml up to 1,000 μg/ml. Modified strains generated by gene trapping were genetically stable in the absence of selective pressure, maintaining the integrated DNA as well as the relative resistance to hygromycin B over a period exceeding 18 months. This represents hundreds of cell divisions where the genetic stability of the trapped gene locus in addition to the expression of the transgene has been maintained in the respective strains. In contradistinction, over the same time period, transgenic Chlamydomonas reinhardtii strains containing hygromycin resistance conferring transgenes under the control of a more conventional Hsp70A-RbcS2 promoter combination, lost or silenced the transgene at a frequency exceeding 50% of the integrant strains. Thus, in Chlamydomonas reinhardtii and most probably in other microalgal strains, the stability of transgenes expressed under the control of trapped endogenous promoter and regulatory sequences far exceeds that of transgenes produced under a more conventional promoter-open reading frame-terminator (3′UTR) construct design. This comparison clearly illustrates benefits provided by employing embodiments of the present invention.

The results are shown in Table 1, below, which provides information regarding the identified site of integration of a selected set of modified strains as well as an identity of the trapped gene where this was identified. This Table 1 also provides information regarding the relative Hygromycin B resistance following gene trapping; it should be noted that a common feature of the gene trapping approach is the identification of modified strains across a wide spectrum of relative antibiotic resistance. This includes strains that are capable of growing under selective conditions that are as much as 10 to 20 times the original antibiotic concentration used to select modified strains. For comparative purposes, conventionally generated transgenic Chlamydomonas strains where the DNA has been inserted randomly into the algal genome and the selectable marker gene is under the control of the preferred Hsp70A-RbcS2 combined promoter in concert with the RbcS2 intron 1 and the RbcS2 3′UTR are only rarely able to grow on selective media that contains 5 times the original antibiotic concentration used to select modified strains. Again, trapped integrant strains consistently out-performed integrant strains produced using the more conventional Hsp70A-RbcS2 combined promoter, commonly used to express transgenes in the nucleus of Chlamydomonas reinhardtii, with a similar strategy employed in most transgenic microalgal strains; such improved performance is a benefit provided by embodiments of the present invention.

TABLE 1
Gene trap integration sites and hygromycin B resistance (μg/ml) following
promoter/polyA gene trapping scenario in Chlamydomonas reinhardtii

| Clone No. | Chromosome No. | Gene/sequence trapped | Hygromycin B resistance |
|---|---|---|---|
| 3.2 | 12 | Hypothetical protein gene ID: 5719715 | 100 |
| 3.5 | 3 | Hypothetical protein: Cre03.g166500 | 100 |
| 4.2 | 12 | NADH: ubiquinone oxidoreductase B14 | 1,000 |
| 4.3 | 11 | Vacuolar sorting protein 11 C terminal | 250 |
| 4.4 | 14 | Assembled EST match (no introns) | 100 |
| 5.2 | 6 | Assembled EST match, insertion in 3′UTR | 100 |
| 6.2 | 12 | Hypothetical protein | 50 |
| 8.1 | 1 | 60S ribosomal protein 11 | 250 |

### Example 2

### Microalgal polyA Gene Trap in Chlamydomonas reinhardtii

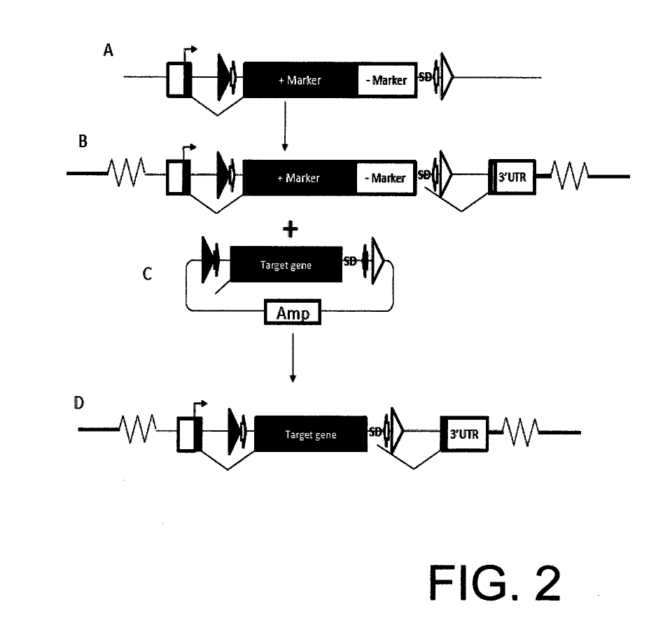

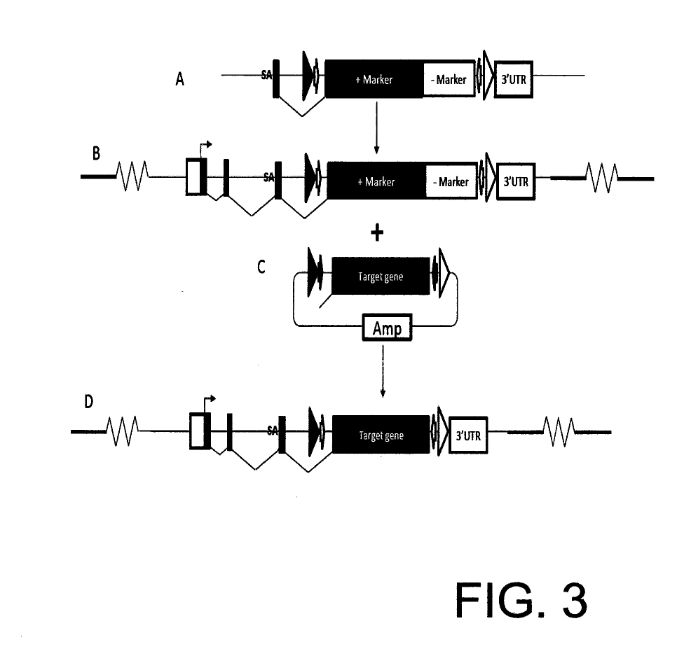

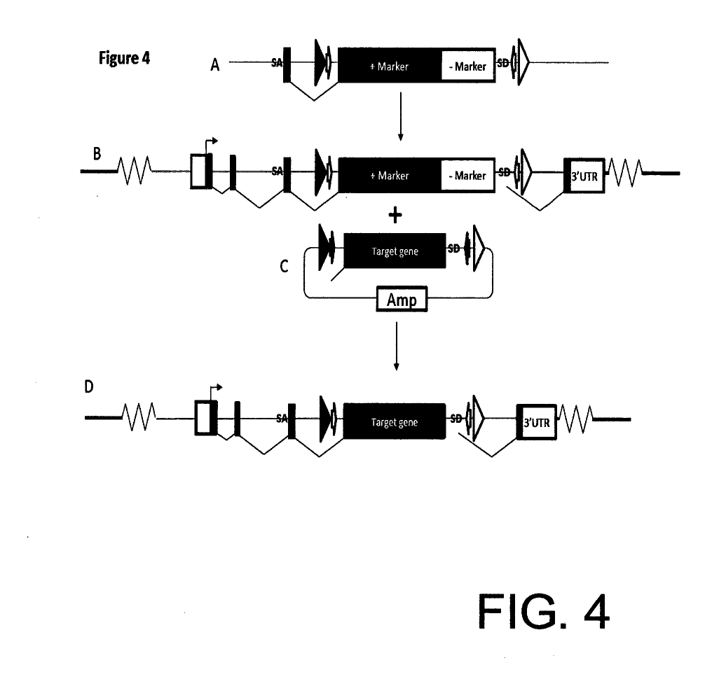

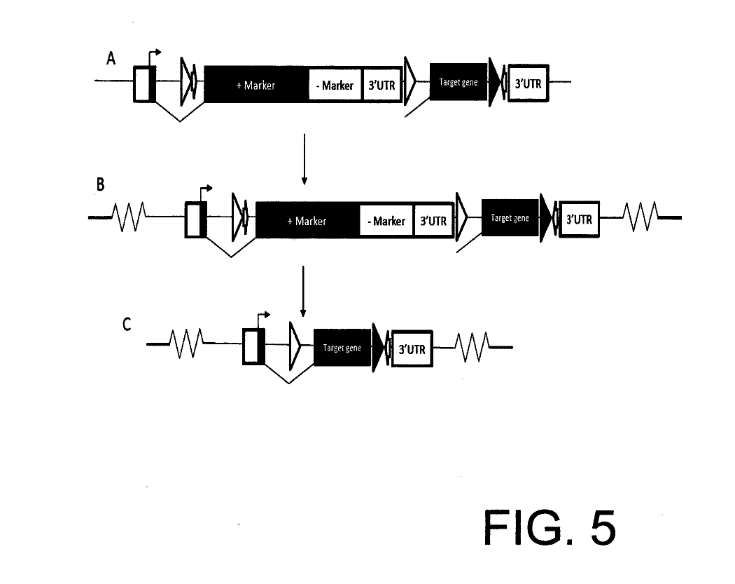

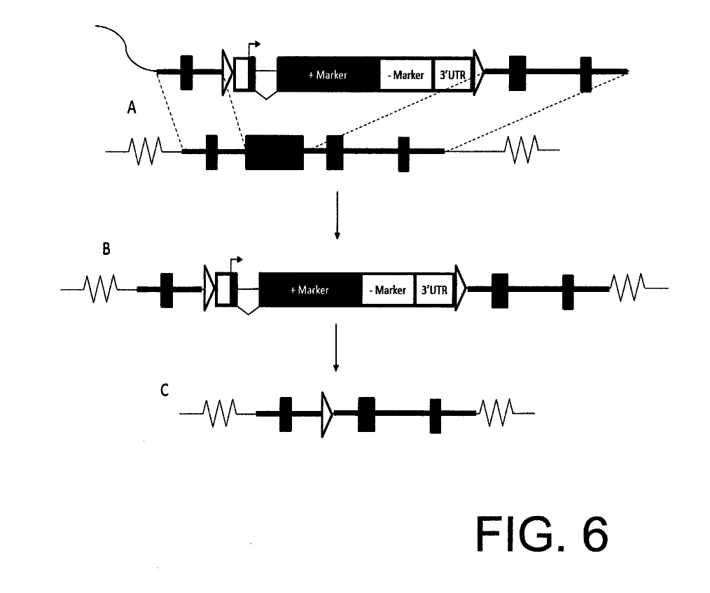

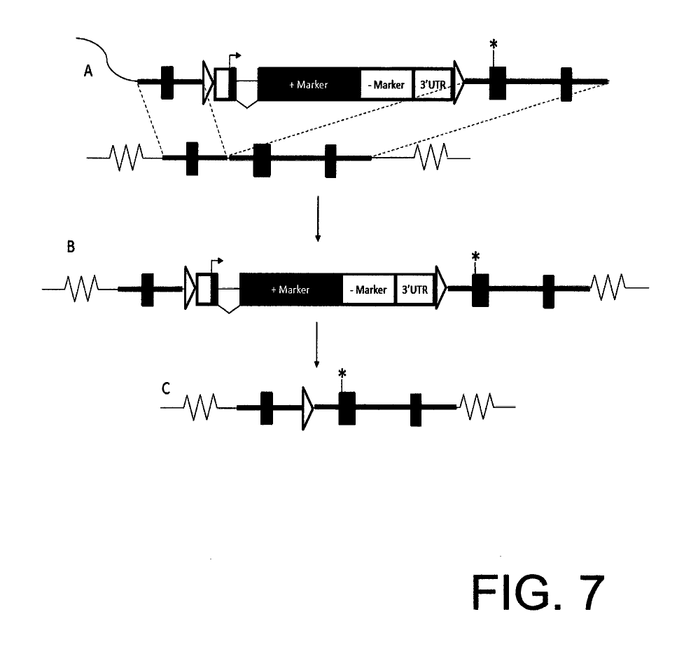

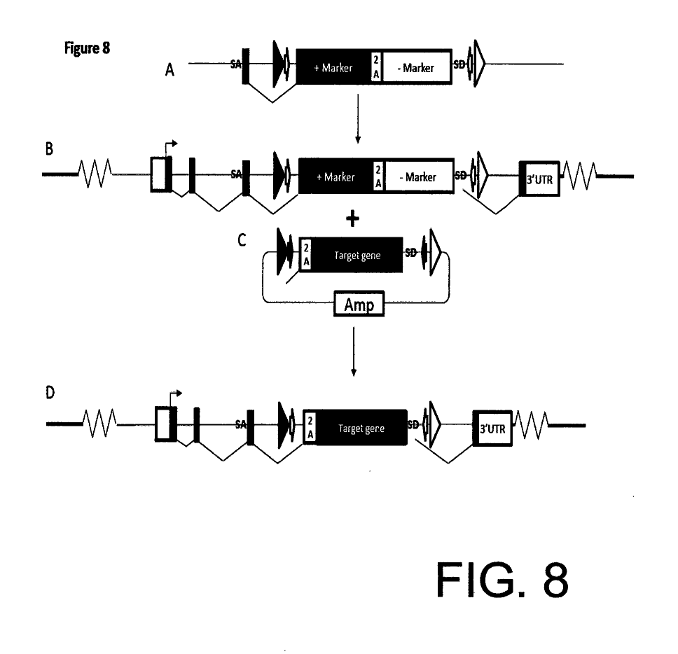

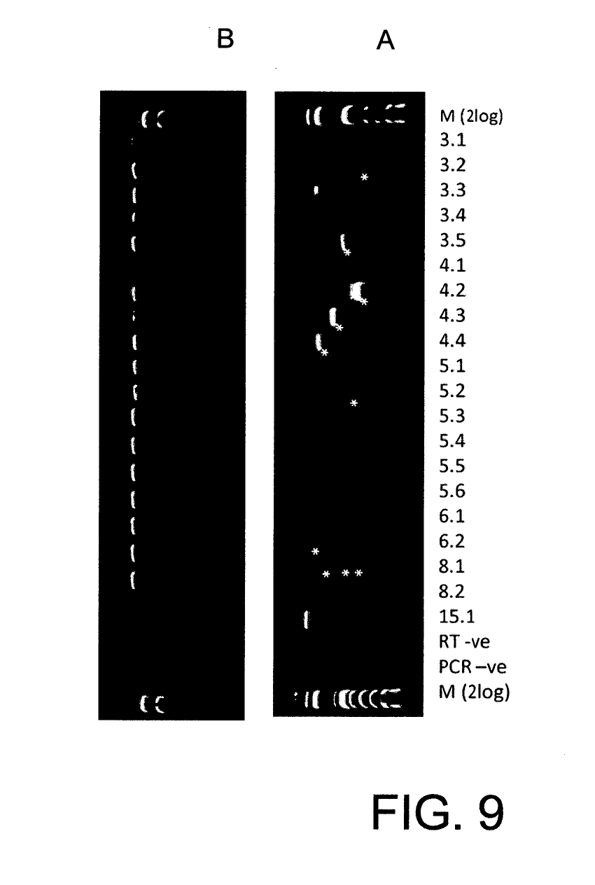

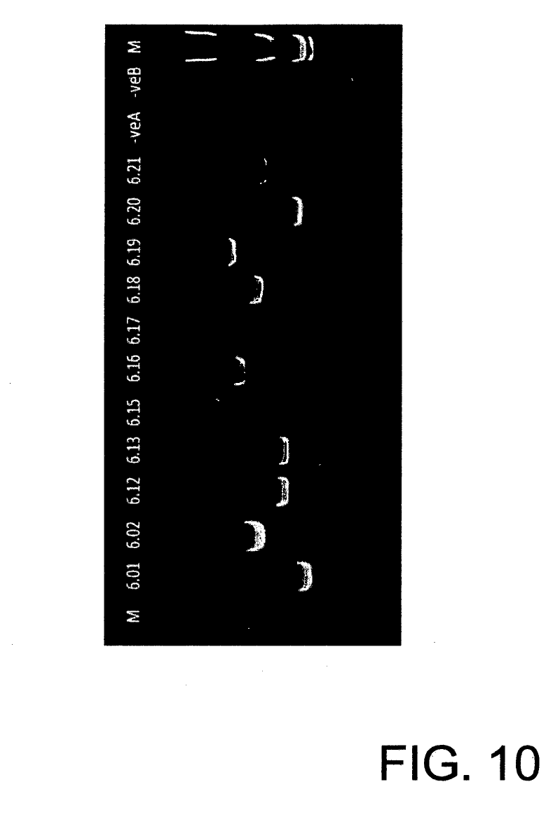

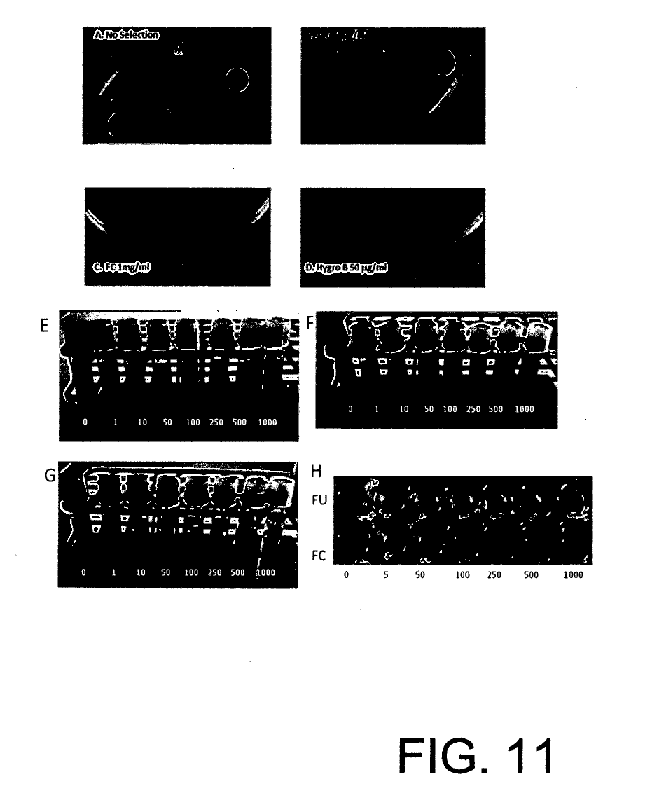

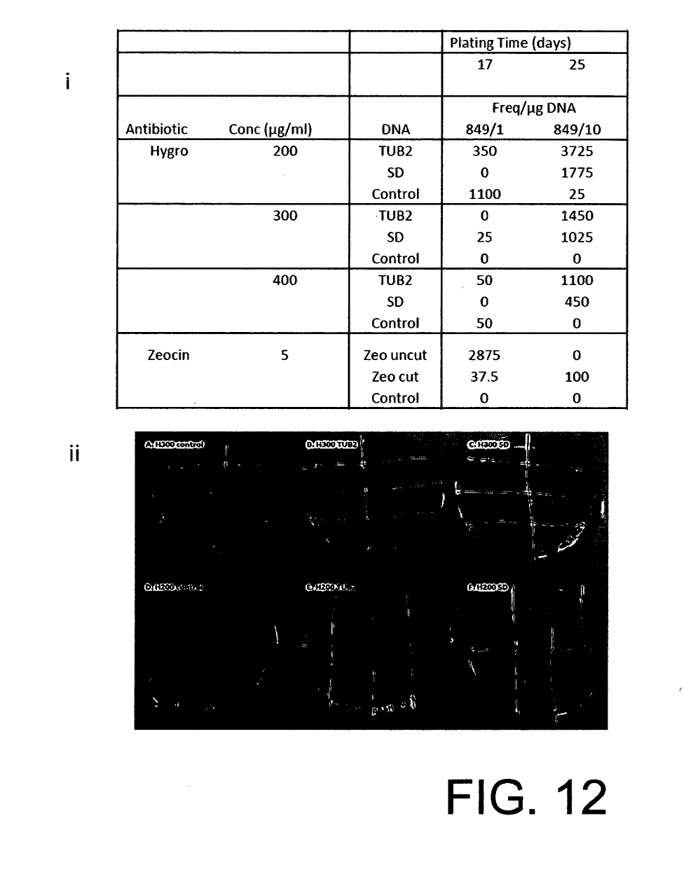

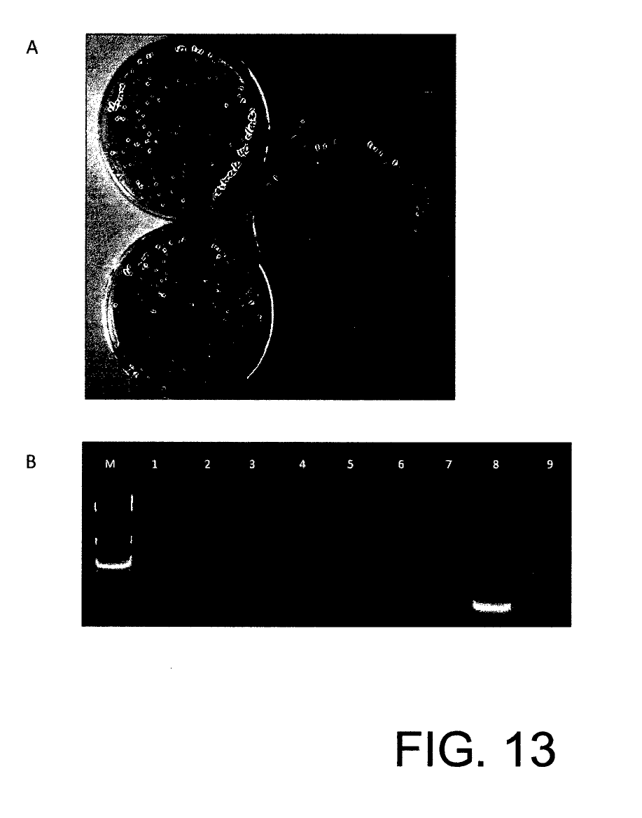

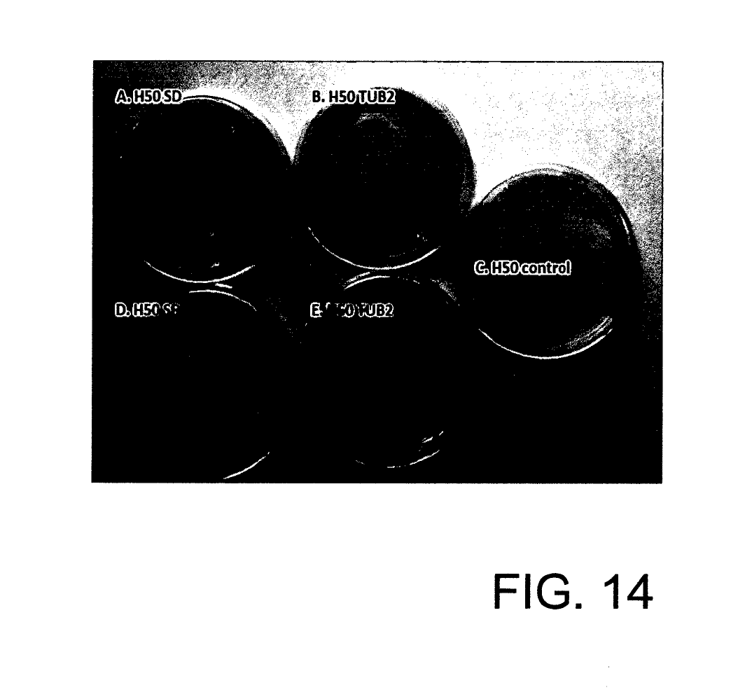

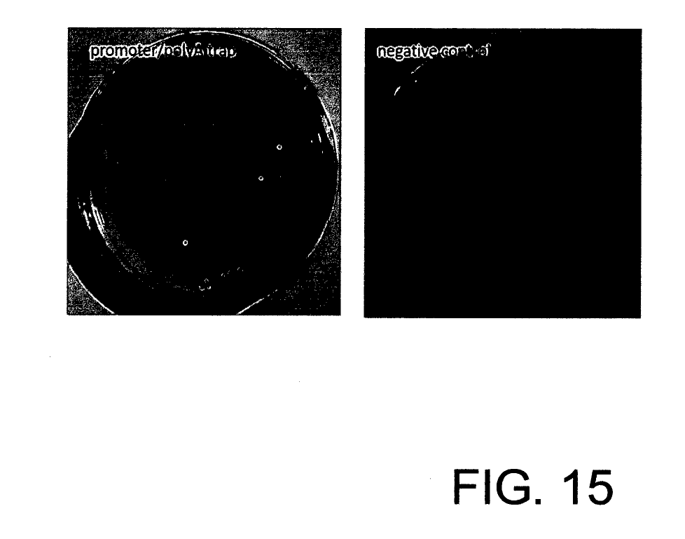

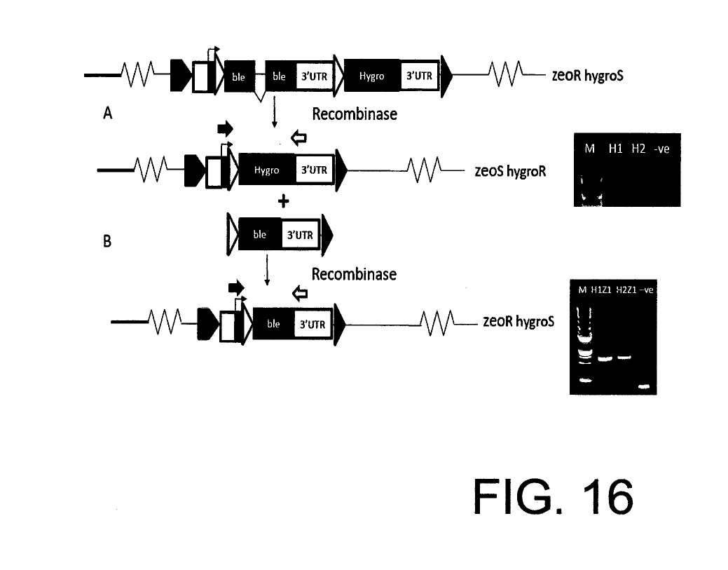

A microalgal polyA gene trap was performed in Chlamydomonas reinhardtii in accordance with the scheme shown in FIG. 2. The polyA trap included the combined Hsp70A and RbcS2 promoters as well as RbcS2 intron 1. The vector was linearised with BamHI, prior to electroporation into Chlamydomonas reinhardtii CC849 cells as described above. Gene trap integrant strains were selected on hygromycin B containing TAP agar plates and were analysed as described above. The frequency of gene trapping events was increased as opposed to the frequency of resistant colonies obtained from the combined promoter/polyA trap. The polyA trap technically does not depend upon trapping of an actively expressed gene as its success depends upon trapping a functional 3′ untranslated sequence or sequence that is able to act as a functional poly-adenylation signal. Between 3 to 5 fold more colonies were routinely observed when comparing the polyA trap approach with the combined promoter/polyA trap.

PolyA trap integrant strains were analysed by 3′RACE to determine the site of insertion. The majority of integrant strains were found to have trapped known genes within the 3′UTR of these genes.

Of significance, it is to be noted that the range of antibiotic concentration over which trapped integrant strains were able to grow was more restricted than that routinely observed when promoter/polyA trap integrant strains were analysed, with the highest concentration of hygromycin at which resistant integrant strains were able to grow typically being 250 μg/ml or less. This was even more so the case with transgenic Chlamydomonas created by transforming algal cells with expression vectors consisting of the Hsp70A and RbcS2 promoter plus intron 1 of RbcS2, hygro gene and the RbcS2 3′ UTR. It was rare to observe any resultant integrant strains that are able to grow robustly in culture medium exceeding 100 μg/ml hygromycin B. Such a result indicates a relative merit of driving transgene expression from endogenous promoter/poly combinations in situ within the algal genome versus the use of exogenous promoters and or 3′UTR sequences that are then inserted at random.

### Example 3

### Microalgal Promoter Trap in Chlamydomonas reinhardtii

A microalgal promoter trap was performed according to the scheme illustrated in FIG. 3. Transformation conditions were as described above. A hygromycin promoter trap construct, was restriction endonuclease digested with. Baran and the specific insert DNA purified by gel extraction using standard procedures. Between 1 to 4 μg purified insert DNA was electroporated into Chlamydomonas cells as described above. Hygromycin B resistant strains were selected as described. An average implementation of embodiments yielded in a range of 50 to several hundred hygromycin B resistant colonies per electroporation. Resistant colonies were restreaked as aforementioned and were analysed for insertion site using two alternate promoter walking PCR-based strategies.

In overview, genomic DNA was extracted from individual integrant strains using described methods (Newman, S. M., Boynton, J. E., Gillham, N. W., Randolph-Anderson, B. L., Johnson, A. M., Harris, E. H. (1990) Transformation of chloroplast ribosomal RNA genes in Chlamydomonas: molecular and genetic characterization of integration events. Genetics, 126(4): 875-88). Five micrograms of each genomic DNA sample was digested by restriction endonuclease digestion using 10 units of SacII restriction endonuclease under standard conditions. Digested DNAs were purified by single phenol:chloroform:isoamyl alcohol extractions followed by ethanol precipitation. After quantitation, 0.5 μg of each genomic DNA sample was ligated in a total volume of 10 μl overnight at 16° C. to 20° C. using T4 DNA Ligase. Two microliters of each sample were used as the template for nested PCR reactions using the following PCR primers. The first PCR reaction ran through 40 cycles using Q5 Hot Start DNA polymerase (New England Biolabs) under recommended reaction conditions. One microliter of each first round PCR reaction was used as the template for the nested PCR reaction, which ran for 35 cycles. Amplified products were analysed by agarose gel electrophoresis, is illustrated in a representative gel image shown in FIG. 10. Strains analysed in this manner, consistently yielded single amplified PCR products, indicating single copy transgene insertion events. Fragments were purified by gel-extraction and nucleotide sequences determined by automated DNA sequencing using the forward nested primer as sequencing primer. The identity of trapped loci was determined using BLAST alignments using either searches of the NCBI or Phytozome (Chlamydomonas reinhardtii current genome release) databases.

The second method employed to determine the site of transgene integration was a nested PCR-based promoter walk employing a degenerate anchor primer in combination with gene-specific primers. Degenerate anchor primers had the following sequences, priming, respectively, on either truncated SacII (5′GCGG3′) (SEQ ID NO:34), EagI (5′GCCG3′) (SEQ ID NO:35) or SmaI/XmaI (5′CGGG3′) (SEQ ID NO:36) restriction endonuclease sites.

Roughly in a range of 50 to 100 ng genomic DNAs isolated from individual gene trap integrant strains were used as the template in nested PCR reactions. Q5 Hot Start polymerase was used under the manufacturer's recommendations using the High-GC enhancer. PCR cycle conditions were 98° C. 10 seconds followed by 68° C. for 1 minute for 40 cycles. One microliter of the first round PCR reactions was used as the template for the second round PCRs, which utilised a nested gene specific PCR primer in combination with the anchor primer, derived from the terminal 25 nucleotides of the degenerate anchor primers previously described. PCR reactions proceeded at the same temperature and cycle lengths as the first round PCRs but through 35 rounds only. Amplified products were visualised through agarose gel electrophoresis and were extracted by gel purification for analysis by automated DNA sequencing.

### Example 4

### Microalgal Promoter Trap with Demonstration of Positive Negative Selection Strategy in Chlamydomonas reinhardtii and Sensitivity of Algal Strains to 5-Fluorouracil

A promoter trap construct was created with the codon-optimised ble Coding sequence fused in frame with a codon-optimised cytosine deaminase sequence derived from a fungal cytosine deaminase amino acid sequence. The originating source species that was used in this instance was Scheffersomyces stipitis (SEQ ID NO:37), while Torulaspora delbrueckii (SEQ ID NO:38) was also tested in a later experiment and found to work as efficiently. It is likely that other fungal encoded cytosine deaminase variants will function equally well within the same context and it would be considered well within the abilities of one skilled in the art to create such variants.

Linearised DNAs were electroporated into Chlamydomonas reinhardtii CC849 cells as described above. Electroporated cells were plated onto selective plates containing 10 μg/ml zeocin to select for those cells that had stably integrated the transgene DNA. Positive integrant strains were restreaked onto selective plates containing zeocin and then established as 10 ml liquid cultures containing 5 μg/ml zeocin. Five microliters of liquid culture derived from three independent integrant strains were spotted onto TAP agar plates containing the following (FIG. 11):

A) no selective agent;
B) 10 μg/ml zeocin;
C) 1 mg/ml fluorocytosine; or
D) 50 μg/ml hygromycin B. Cultures were grown under constant illumination at a temperature in a range of 26° C. to 28° C. for 28 days before scoring for growth.

Transgenic strains for ble-codA grew on non-selective medium and medium containing 10 μg/ml zeocin, but failed to grow on plates containing 1 mg/ml fluorocytosine or 50 μg/ml hygromycin B; see FIG. 11.

Flurouracil would be the product of cytosine deaminase action on fluorocytosine. To be valid as a generally applicable negative selection strategy, fluorocytosine must be non-toxic and non-inhibitory to microalgal cell growth whereas fluorouracil must be toxic and inhibitory to microalgal cell growth. In order to determine the relative validity of the application of codA as a general purpose negative selectable marker in microalgae, the following microalgal strains (1 to 5×10⁶ cells/ml starting culture densities) were cultured in an ascending series of culture medium supplemented with 5-fluorouracil at 0, 5 (or 1), 50, 100, 250, 500 and 1,000 μg/ml 5-fluorouracil versus the same amount of fluorocytosine:

Chlorella (Auxenochlorella) protothecoides CCAP 211/8D; Chlorella vulgaris (UK native strain: 4TC3/16); Chlorella sorokiniana (UTEX1230); Chlamydomonas reinhardtii CC849; Dunaliella salina CCAP 19/30; Haematococcus pluvialis (UK native strain LSBB312); Nannochloropsis oculata CCAP 849/1; Nannochloropsis oceanica CCAP 849/10 Ostreococcus tauri.

Fluorouracil was demonstrated to be toxic in a dose dependent manner whereas fluorocytosine was found to be non-toxic; see FIG. 11.

### Example 5

### Microalgal Promoter and Promoter/polyA Trap in Nannochloropsis Strains

To demonstrate the applicability and validity of the above described gene trapping platform in species other than Chlamydomonas, Nannochloropsis oculata CCAP 849/1 and Nannochloropsis oceanica CCAP 849/10 strains were transformed with the BamHI cut and gel purified hygro promoter trap vector (TUB2) and the hygro promoter/polyA trap vector (SD). Electroporation conditions were modified from Kilian, O., Benemann, C. S. E., Niyogi, K. K. And Vick, B. (2011) High-efficiency homologous recombination in the oil-producing alga Nannochloropsis sp. Proc. Natl. Acad. Sci. USA 108 (52):21265-21269. For each strain, starter cultures growing in F/2 culture medium, with ammonium in place of nitrate, at late-log phase were used to inoculate 300 mL cultures in the same media, at a concentration in a range of 5 to 10×10⁵ cells/mL. Growth conditions were a temperature of 20° C., 16 hr Light/8 hr Dark, in a range of 80 to 100 mot photons per m² per sec in vertical tubes with 2% CO₂ supplied for N. oceanica CCAP 849/10 and air for N. oculata CCAP 849/1. Cells were grown to 3 to 6×10⁶ cells/mL, harvested by centrifugation (5500×g, 7 min), resuspended and washed 6 times sequentially (recentrifuging each time) in 0.4M D-sorbitol. The pellet was resuspended in 0.4 M Sorbitol to adjust cells to ˜10¹⁰/mL and 50 μl aliquots were mixed with 1 μL of DNA (1 μg/μL) and electroporated using a 1 mm gap cuvette, single pulse with field strength 22 kV/cm, 10 ρF capacitance. Cells were immediately transferred to 10 ml F/2 medium and recovered for a period of 1 to 3 days at a low light at a temperature of 20° C. Aliquots of 400 μL were spread on selective media (F/2 agar+antibiotic) on 90 mm plates. Plates were incubated for a period of 3 to 4 weeks under the aforesaid conditions. Cultures were selected by growth on agar plates containing either 200, 300 or 400 micrograms/ml hygromycin B. Control plates were represented by electroporations that received no DNA for each test. As a point of comparison, a VCP1 promoter-ble-terminator (zeocin resistance) plasmid was electroporated as either linearised plasmid DNA or uncut, intact plasmid. FIG. 12 provides an illustration of N. oceanica CCAP 849/10 plates 25 days after plating on selective plates, Hygro resistance colonies were also obtained in Nannochloropsis oculata, but at a reduced frequency as indicated in a table shown in FIG. 12. Resistant integrant strains were restreaked onto selective plates to confirm and then analysed for insertion site using an equivalent approach described previously for Chlamydomonas.

### Example 6

### Microalgal Promoter and Promoter/polyA Trap in Chlorella sp Strains

In order to investigate the utility of the gene trap and gene replacement strategy in Chlorella strains, DNAs representing the gene trapping constructs described above in successful electroporation-based transformation tests in Chlamydomonas and Nannochloropsis strains were transferred using standard molecular biology approaches into an Agrobacterium binary vector backbone derived from pCAMBIA2300 between the Agrobacterial vector left and right border sequences such that all but 100 bp of the previous binary vector sequence was replaced. Hygro and nptII variants of the gene trap vectors utilised above were created in the pCAMBIA2300 backbone. Versions with a Chlamydomonas TUB2 3′UTR (promoter trap only) or the splice donor (SD) version (combined promoter/polyA trap) were created in each instance. DNAs were transformed into the Agrobacterium binary strain, LBA4404 using electroporation under conditions recommended for this strain. Agrobacterium cells resistant to both kanamycin and streptomycin (50 μg/ml and 100 μg/ml, respectively) were selected, representing Agrobacterial strains carrying the gene trapping construct within a binary vector host.

Approximately 1×10⁶ late logarithmic to stationary phase Chlorella vulgaris (UK native strain 4TC3/16) or Chlorella sorokiniana UTEX 1230 strain cells were spread on TAP-agar plates supplemented with acetosyringone to a final concentration of 100 μM for 48 hours at a temperature in a range of 26° C. to 28° C. under a constant illumination in a range of 150 to 200 μmol/m²/s. Agrobacterial cultures carrying the gene trap plasmids were cultured at 28° C. in YEP medium supplemented with kanamycin and streptomycin (50 and 100 μg/ml, respectively) for 48 hours with constant shaking at 250 rpm. Agrobacterium were diluted to an OD600 value in a range of 0.4 to 0.6 in a TAP medium (with acetate) supplemented with acetosyringone to 100 μM. Two hundred microliters of Agrobacterium suspension were spread onto individual plates of Chlorella cultures using sterile glass spreaders. Plates were allowed to dry then sealed with Parafilm and cultured for 48 hours (vulgaris) or 24 hours (sorokiniana) in low light, at a temperature of 28° C. After the respective amounts of time, 1 milliliter of the TAP medium supplemented with cefotaxime to 500 μg/ml was added to individual plates and the cell suspensions gently scraped into the solution before 200 microliter aliquots of each suspension were spread onto individual plates containing either G418 (for nptII gene trap constructs or as a negative control with cells co-cultivated with the Agro hygro gene trap constructs) or hygromycin B (for hygro gene trap constructs or as a negative control with cells co-cultivated with the Agro nptII gene trap constructs). Vulgaris was spread on 90 mm TAP-agar plates containing either 25 μg/ml G418 or 100 μg/ml hygromycin B, whereas sorokiniana was spread on plates containing either 200 μg/ml G418 or 1 mg/ml hygromycin B. After plates were allowed to dry they were sealed with parafilm then cultured under constant illumination (150 to 200 μmol/m2/s) and at a temperature range of 26° C. to 28° C. for a period of 5 to 14 days. Resistant colonies started to appear after a period in a range of 5 to 8 days for both strains. Negative controls in each instance represented cells co-cultivated with the alternate selectable marker—for instance cells co-cultivated with the nptII gene trap strain were plated on hygromycin B containing plates to provide a negative control for the hygro gene trap co-cultivations and vice versa. Relative numbers of colonies obtained on the negative control plates were used as an indicator of the background levels of antibiotic resistance observed in each respective strain. Typical plates obtained from promoter trapping in Chlorella vulgaris are shown in FIG. 13.

Antibiotic resistant colonies, representing supposed gene trap integrant strains for the two Chlorella strains were restreaked onto 90 mm TAP agar plates containing G418 or hygromycin B: Chlorella vulgaris, 30 μg/ml G418 and 150 μg/ml hygromycin. B; Chlorella sorokiniana, 250 μg/ml G418 and 1.2 mg/ml hygromycin B. Integrant strains that grew under these conditions were processed for DNA analyses under conditions previously described for Chlamydomonas. The presence of the transgene DNA was initially confirmed in supposed transformed Chlorella integrant strains by PCR amplification with gene specific primers using quick DNA preps as PCR template. A result showing the confirmation of two vulgaris and two sorokiniana gene trap integrant strains is illustrated in FIG. 13.

### Example 7

### Microalgal Promoter and Promoter/polyA Trap in Haematococcus pluvialis

Haematococcus pluvialis, widely cultivated to produce the potent antioxidant, astaxanthin, was successfully transformed using a similar Agrobacterium co-cultivation method as described for Chlorella strains above. In overview, Haematococcus pluvialis cells were cultured in TAP medium (containing acetate) supplemented with vitamins B1 (thiamine hydrochloride) and B12 (cyanocobalamin) to 4.5 μM and 0.74 μM, respectively under a 16 hr light/8 hour dark diurnal light profile peaking at 100 μM/m²/s light intensity. Four hundred milliliter cultures were established using the Algem® labscale photobioreactor (Algenuity, Stewartby, Beds, UK), with cell cultures harvested at 3×10⁵ cells/ml at mid-logarithmic green phase. Cells were pelleted and spread (1×10⁶ cells/plate) on TAP-agar+vitamins 90 mm plates supplemented with 100 μM acetosyringone. Cells were cultured for 48 hours under constant illumination at <50 μM/m²/s illumination at 22° C. to 24° C. Agrobacterium co-cultivation was carried out as performed for Chlorella strains except, co-cultivated cells were ultimately spread on single selective TAP-agar+vitamins (G418, 10 μg/ml or hygromycin B, 50 μg/ml) plates and cultivated under low light (<50 μM/m²/s) at 22° C. to 24° C. for a period in a range of 7 to 14 days. Negative control plates were produced as described for Chlorella strains with nptII co-cultivated cells plated on hygro containing plates acting as the negative control for the hygro constructs and vice versa. Presumed resistant cells were restreaked on TAP-agar+vitamin, selective plates and also established as liquid cultures using G418 (10 μg/ml) or hygromycin B (50 μg/ml). An experiment illustrating successful derivation of hygromycin resistant cells from Agrobacterium mediated transformation of Haematococcus pluvialis cells with a hygro promoter trap construct is shown in FIG. 14.

### Example 8

### Microalgal Promoter and Promoter/polyA Trap in Phaeodactylum tricornutum

To assess the broad applicability of the gene trapping platform in microalgal strains outside the Chlorophyta, we created promoter and polyA trap constructs incorporating a ble expression cassette (as described in Zaslayskaia, L. A., Lippmeier, J. C., Kroth, P. G., Grossman, A. R. and Apt, K. E. (2000) Transformation of the diatom Phaeodactylum tricornutum (Bacillariophyceae) with a variety of selectable marker and reporter genes. J. Phycol. 36, 379-386) combined with or without the Chlamydomonas reinhardtii RbcS2 intron 1 sequence (SEQ ID NO:21) and with or without the Phaeodactylum tricornutum fcpA terminator (3′UTR) sequence (SEQ ID NO: 39). Intact plasmid DNAs were introduced into Phaeodactylum tricornutum cells using biolistics, essentially as described in Zaslayskaia et al, except 0.9×10⁷ cells were treated. Negative control plates represent cells that were not treated by biolistics. Cells were selected by growth on 9 cm artificial seawater 1.2% agar plates containing 100 μg/ml zeocin and incubated at 20° C. constant temperature and 75 μmol/m²/s constant illumination. Colonies were scored at between 10 to 21 days of growth and were restreaked onto selective plates. A typical result obtained from transformation of Phaedactylum tricornutum cells (0.9×10⁷ cells) with the promoter or promoter/polyA gene trapping constructs is shown in FIG. 15.

### Example 9

### Microalgal Recombinase Dependent Marker Gene Deletion or Replacement

To assess the utility of site-specific recombinases in microalgal strains, first, plasmid reporter constructs were made for application in Chlamydomonas reinhardtii (organisation of the constructs and the process of converting from one marker to another is shown in FIG. 16). A construct incorporating a ble expression cassette under the control of the combined Hsp70A-RbcS2 promoter and the RbcS2 terminator sequence positioned immediately upstream of an open reading frame encoding a codon optimised hygro cassette and TUB2 3′UTR (FIG. 16). The ble-3′UTR cassette was flanked by direct copies of the wild-type FRT sequence, with the first of the two copies inserted in-frame immediately following the translation initiation codon. The second of the two copies was positioned at the beginning of the hygro open reading frame such that a Flp-dependent deletion event would result in specific deletion of the ble-3′UTR cassette and placement of the hygro open reading frame into position immediately following the translation initiation codon. The presence of the ble-3′UTR cassette effectively acts to stop transcription of the hygro sequence. Only if ble-3′UTR is precisely removed in a Flp-dependent manner will hygro expression occur with the resultant cells being converted from hygro-sensitive (hygroS) to hygro-resistant (hygroR). The initial construct also contains a third FRT site, this time a FRT3 site, positioned downstream of the hygro-3′UTR cassette.

Ble resistant cells were produced by electroporation of Chlamydomonas reinhardtii CC849 using conditions as described above with linearised plasmid DNAs. Cells were selected on TAP-agar plates containing 10 μg/ml zeocin. Ble resistant strains were restreaked on zeo plates and then spotted onto TAP agar plates containing 50 μg/ml hygromycin B to confirm sensitivity to hygromycin B. Ble resistant clones were expanded into liquid culture and transformed with an algal expression construct containing a Flp recombinase open-reading frame with added nuclear localisation signal. Electroporated cells were plated onto TAP-agar plates containing 50 μg/ml hygromycin B. Resistant colonies were observed after 6 to 9 days and were restreaked onto TAP-agar hygro plates to confirm resistance to hygromycin B. Cells were also spotted onto TAP-agar ble plates to confirm sensitivity to zeocin. Genomic DNAs were prepared from strains and the presence of the correctly recombined transgene was confirmed by PCR amplification and automated sequencing of the PCR products. FIG. 16 shows PCR results from two hygromycin B resistant clones (H1 and H2) as well as a negative control (parental CC849 cell line).

Lastly, strains containing transgenes conferring hygromycin B resistance were converted, back to strains that were resistant to zeocin through a gene replacement strategy. A promoterless ble-3′UTR DNA lacking any splice acceptor or donor sequences, but flanked at the 5′ end by a wild-type FRT sequence and at the 3′ end by a FRT3 sequence, was transformed into hygro resistant cells produced above along with the Flp recombinase expression vector described above. Electroporated cells were plated onto TAP-agar plates containing 10 μg/ml zeocin and zeo resistant colonies derived. Resistant colonies were restreaked onto zeo plates and the structure of the correctly recombined transgene was confirmed using PCR and automated sequencing. FIG. 16 shows PCR results from two zeocin resistant clones (H1Z1 and H2Z1) generated from two original hygromycin B resistant clones by recombinase-dependent gene replacement, demonstrating the utility of this approach for gene replacement in microalgae.

1-55. (canceled)

56. A method of modifying an algal cell genome, wherein said method comprises steps of:
providing an algal cell;
introducing into said algal cell an integration cassette comprising at least two site-specific recombination sites and at least one selectable marker gene, wherein the at least two site specific recombination sites are positioned to flank the at least one selectable marker gene; and
selecting cells which have incorporated the integration cassette by cultivating the cells in a selective media and selecting growing cells, wherein an ability of the cells to be cultured on the selective media is dependent on a presence of the at least one selectable marker in a genome of the algal cell.

57. The method of claim 56, wherein the method includes arranging for the selected cells to incorporate the integration cassette within an actively expressed gene thereof.

58. The method of claim 56, wherein the method includes, for the integration cassette, utilizing an algal promoter sequence positioned upstream of (5′ to) the at least one selectable marker and upstream of (5′ to) the site specific recombination site.

59. The method of claim 58, wherein the method includes employing an untranslated region (UTR) upstream of the promoter (5′UTR).

60. The method of claim 58, wherein the method includes employing an intron splice donor sequence downstream of (3′ to) the at least one selectable marker and upstream of (5′ to) the other downstream recombination site.

61. The method of claim 58, wherein the method includes utilizing constitutive algal promoter or engineered combinations thereof.

62. The method of claim 61, wherein the method includes selecting the constitutive algal promoter from a group consisting of a Hsp70A promoter (SEQ ID NO:5), the RbcS2 promoter (SEQ ID NO):6) and a beta-2-tubulin (TUB2) promoter (SEQ ID NO:7).

63. The method of claim 56, wherein the method further includes, for the integration cassette, employing a 3′ untranslated region (3′ UTR) sequence downstream of (3′ to) the at least one selectable marker and downstream of (3′to) the site specific recombination site.

64. The method of claim 63, wherein the method includes employing an intron splice acceptor sequence upstream of (5′ to) the at least one selectable marker and upstream of (5′ to) the upstream recombination site.

65. The method of claim 59, wherein the method includes obtaining the untranslated region (UTR) from an algal gene.

66. A method for introducing a target gene sequence into a primed algal cell, wherein the method includes steps of:
supplying a primed algal cell by:
providing an algal cell;
introducing into said algal cell an integration cassette comprising at least two site-specific recombination sites and at least one selectable marker gene, wherein the at least two site specific recombination sites are positioned to flank the at least one selectable marker gene; and
selecting cells which have incorporated the integration cassette by cultivating the cells in a selective media and selecting growing cells, wherein an ability of the cells to be cultured on the selective media is dependent on a presence of the at least one selectable marker in a genome of the algal cell;
wherein the algal cell comprises a type I site-specific recombination site and a type II site specific recombination site, wherein the type I site-specific recombination site is different from the type II site-specific recombination site such that it is heterospecific and does not recombine with the type II site-specific recombination site, within the algal cell genome;
providing a target cassette comprising a target gene sequence flanked by a type I site specific recombination site and a type II site-specific recombination site such that these sequences are capable of recombining with the recombination sites in the primed algal cell; and
effecting targeted site-specific recombinase mediated insertion of the target cassette into the algal genome by effecting recombination between corresponding type I and type II site-specific recombination sites flanking the target gene sequence and located in the algal genome, such that the target gene sequence is introduced into the algal genome.

67. The method of claim 66, wherein the type I site-specific recombination site and the type II site-specific recombination site are within an actively expressed gene of the algal cell genome.

68. The method of claim 66, wherein the target gene sequence comprises two or more target genes.

69. The method of claim 68, wherein the two or more target genes are separated by a sequence encoding self-cleaving 2A peptides.

70. The method of claim 67, wherein the type I site-specific recombination site and the type II site-specific recombination site located in the algal genome are positioned to flank the at least one selectable marker gene.

71. The method of claim 70, wherein the at least one selectable marker gene is a positive selectable marker gene.

72. The method of claim 71, wherein the positive selectable marker gene confers resistance to an antibiotic selected from the group consisting of hygromycin B (such as a hph gene), zeocin (such as a ble gene), kanamycin or G418 (such as a nptII or aphVIII gene), spectinomycin (such as a aadA gene), neomycin (such as a aphVIII gene) or paromomycin (such as a aphVIII gene) or a herbicide selected from a group consisting of phosphinothricin and norflurazon.

73. The method of claim 71, wherein the integration cassette further comprises a negative selectable marker gene.

74. The method of claim 73, wherein the negative selectable marker gene is fused in-frame with the positive selectable marker gene.

75. The method of claim 73, wherein the positive selectable marker gene and the negative selectable marker gene are separated by a sequence encoding a self-cleaving 2A peptide.

76. A modified algal cell produced by:
providing an algal cell;
introducing into said algal cell an integration cassette comprising at least two site-specific recombination sites and at least one selectable marker gene, wherein the at least two site specific recombination sites are positioned to flank the at least one selectable marker gene; and
selecting cells which have incorporated the integration cassette by cultivating the cells in a selective media and selecting growing cells, wherein an ability of the cells to be cultured on the selective media is dependent on a presence of the at least one selectable marker in a genome of the algal cell.
